SEQUENCE LISTING (202 sequences)

SEQ ID NO: 1 - RNA, 22 nt, Homo sapiens
uuagggcccuggcuccaucucc

SEQ ID NO: 2 - RNA, 21 nt, Homo sapiens
caaaacgugaggcgcugcuau

SEQ ID NO: 3 - RNA, 22 nt, Homo sapiens
cagcagcaauucauguuuugaa

SEQ ID NO: 4 - RNA, 21 nt, Homo sapiens
uggguuuacguugggagaacu

SEQ ID NO: 5 - RNA, 21 nt, Homo sapiens
ugagaugaagcacuguagcuc

SEQ ID NO: 6 - RNA, 18 nt, Homo sapiens
auccuguucgugacgcca

SEQ ID NO: 7 - RNA, 20 nt, Homo sapiens
gaaucccacuucugacacca

SEQ ID NO: 8 - RNA, 18 nt, Homo sapiens
aucccacuccugacacca

SEQ ID NO: 9 - RNA, 22 nt, Homo sapiens
uuaucagaaucuccagggguac

SEQ ID NO: 10 - RNA, 22 nt, Homo sapiens
uucaccaccuucuccacccagc

SEQ ID NO: 11 - RNA, 19 nt, Homo sapiens
agugaagcauuggacugua

SEQ ID NO: 12 - RNA, 22 nt, Homo sapiens
ucagugcacuacagaacuuugu

SEQ ID NO: 13 - RNA, 23 nt, Homo sapiens
guccaguuuucccaggaaucccu

SEQ ID NO: 14 - RNA, 23 nt, Homo sapiens
uaauacugccggguaaugaugga

SEQ ID NO: 15 - RNA, 22 nt, Homo sapiens
cuugguucagggagggucccca

SEQ ID NO: 16 - RNA, 23 nt, Homo sapiens
aacauucauuguugucggugggu

SEQ ID NO: 17 - RNA, 22 nt, Homo sapiens
gccugcugggguggaaccuggu

SEQ ID NO: 18 - RNA, 22 nt, Homo sapiens
ggagacgcggcccuguuggagu

SEQ ID NO: 19 - RNA, 22 nt, Homo sapiens
aagacgggaggaaagaagggag

SEQ ID NO: 20 - RNA, 22 nt, Homo sapiens
cugaagcucagagggcucugau

SEQ ID NO: 21 - RNA, 24 nt, Homo sapiens
uugcagcugccugggagugacuuc

SEQ ID NO: 22 - RNA, 23 nt, Homo sapiens
uaauccuugcuaccugggugaga

SEQ ID NO: 23 - RNA, 22 nt, Homo sapiens
cuggcccucucugcccuuccgu

SEQ ID NO: 24 - RNA, 25 nt, Homo sapiens
ggcggagggaaguagguccguuggu

SEQ ID NO: 25 - RNA, 22 nt, Homo sapiens
acaggugagguucuugggagcc

SEQ ID NO: 26 - RNA, 22 nt, Homo sapiens
aucaugaugggcuccucggugu

SEQ ID NO: 27 - RNA, 20 nt, Homo sapiens
uaaggcacgcggugaaugcc

SEQ ID NO: 28 - RNA, 23 nt, Homo sapiens
aggaagcccuggaggggcuggag

SEQ ID NO: 29 - RNA, 21 nt, Homo sapiens
acaugaaaaggggagagggca

SEQ ID NO: 30 - RNA, 22 nt, Homo sapiens
uggagaagacuggagaggguau

SEQ ID NO: 31 - RNA, 28 nt, Homo sapiens
cccaggcuggaguguaguggcgugaucu

SEQ ID NO: 32 - RNA, 22 nt, Homo sapiens
aacuggggcgggaagggggaag

SEQ ID NO: 33 - RNA, 24 nt, Homo sapiens
agcagaagcagggagguucuccca

SEQ ID NO: 34 - RNA, 23 nt, Homo sapiens
uggugcggagagggcccacagug

SEQ ID NO: 35 - RNA, 21 nt, Homo sapiens
uggaggagaaggaaggugaug

SEQ ID NO: 36 - RNA, 24 nt, Homo sapiens
cauagcccggucgcugguacauga

SEQ ID NO: 37 - RNA, 23 nt, Homo sapiens
ccaguuaccgcuuccgcuaccgc

SEQ ID NO: 38 - RNA, 22 nt, Homo sapiens
ugagaccucuggguucugagcu

SEQ ID NO: 39 - RNA, 23 nt, Homo sapiens
ugucugcccgcaugccugccucu

SEQ ID NO: 40 - RNA, 23 nt, Homo sapiens
ucggggaucaucaugucacgaga

SEQ ID NO: 41 - RNA, 23 nt, Homo sapiens
aucaacagacauuaauugggcgc

SEQ ID NO: 42 - RNA, 20 nt, Homo sapiens
accaggaggcugaggccccu

SEQ ID NO: 43 - RNA, 22 nt, Homo sapiens
acuccagccccacagccucagc

SEQ ID NO: 44 - RNA, 22 nt, Homo sapiens
uuuccggcucgcgugggugugu

SEQ ID NO: 45 - RNA, 23 nt, Homo sapiens
ugagcgccucgacgacagagccg

SEQ ID NO: 46 - RNA, 22 nt, Homo sapiens
augcaccugggcaaggauucug

SEQ ID NO: 47 - RNA, 22 nt, Homo sapiens
acagcaggcacagacaggcagu

SEQ ID NO: 48 - RNA, 22 nt, Homo sapiens
ugggucuuugcgggcgagauga

SEQ ID NO: 49 - RNA, 22 nt, Homo sapiens
gcugacuccuaguccagggcuc

SEQ ID NO: 50 - RNA, 22 nt, Homo sapiens
ccgcacuguggguacuugcugc

SEQ ID NO: 51 - RNA, 22 nt, Homo sapiens
acugcugagcuagcacuucccg

SEQ ID NO: 52 - RNA, 23 nt, Homo sapiens
cccaguguucagacuaccuguuc

SEQ ID NO: 53 - RNA, 21 nt, Homo sapiens
cuagacugaagcuccuugagg

SEQ ID NO: 54 - RNA, 21 nt, Homo sapiens
ucgaggagcucacagucuagu

SEQ ID NO: 55 - RNA, 22 nt, Homo sapiens
uggagagaaaggcaguuccuga

SEQ ID NO: 56 - RNA, 22 nt, Homo sapiens
aaggagcucacagucuauugag

SEQ ID NO: 57 - RNA, 23 nt, Homo sapiens
ucucacacagaaaucgcacccgu

SEQ ID NO: 58 - RNA, 23 nt, Homo sapiens
uaaggugcaucuagugcaguuag

SEQ ID NO: 59 - RNA, 21 nt, Homo sapiens
aacauagaggaaauuccacgu

SEQ ID NO: 60 - RNA, 22 nt, Homo sapiens
gaauguugcucggugaaccccu

SEQ ID NO: 61 - RNA, 22 nt, Homo sapiens
gaaguuguucgugguggauucg

SEQ ID NO: 62 - RNA, 22 nt, Homo sapiens
aaacaaacauggugcacuucuu

SEQ ID NO: 63 - RNA, 22 nt, Homo sapiens
uuuguucguucggcucgcguga

SEQ ID NO: 64 - RNA, 22 nt, Homo sapiens
uagguuauccguguugccuucg

SEQ ID NO: 65 - RNA, 22 nt, Homo sapiens
ugagaacugaauuccauaggcu

SEQ ID NO: 66 - RNA, 22 nt, Homo sapiens
ugagaacugaauuccauggguu

SEQ ID NO: 67 - RNA, 22 nt, Homo sapiens
agagguaguagguugcauaguu

SEQ ID NO: 68 - DNA, 28 bp, artificial sequence
  Synthetic
cagtcatttggcttagggccctggctcc

SEQ ID NO: 69 - DNA, 21 bp, artificial sequence
  Synthetic
caaaacgtgaggcgctgctat

SEQ ID NO: 70 - DNA, 28 bp, artificial sequence
  Synthetic
cagtcatttggccagcagcaattcatgt

SEQ ID NO: 71 - DNA, 28 bp, artificial sequence
  Synthetic
cagtcatttggctgggtttacgttggga

SEQ ID NO: 72 - DNA, 28 bp, artificial sequence
  Synthetic
cagtcatttggctgagatgaagcactgt

SEQ ID NO: 73 - DNA, 20 bp, artificial sequence
  Synthetic
gcatcctgttcgtgacgcca

SEQ ID NO: 74 - DNA, 22 bp, artificial sequence
  Synthetic
gcgaatcccacttctgacacca

SEQ ID NO: 75 - DNA, 21 bp, artificial sequence
  Synthetic
ggcatcccactcctgacacca

SEQ ID NO: 76 - DNA, 28 bp, artificial sequence
  Synthetic
cagtcatttggcttatcagaatctccag

SEQ ID NO: 77 - DNA, 28 bp, artificial sequence
  Synthetic
cagtcatttggcttcaccaccttctcca

SEQ ID NO: 78 - DNA, 24 bp, artificial sequence
  Synthetic
ttggcagtgaagcattggactgta

SEQ ID NO: 79 - DNA, 28 bp, artificial sequence
  Synthetic
cagtcatttggctcagtgcactacagaa

SEQ ID NO: 80 - DNA, 28 bp, artificial sequence
  Synthetic
cagtcatttggcgtccagttttcccagg

SEQ ID NO: 81 - DNA, 28 bp, artificial sequence
  Synthetic
cagtcatttgggtaatactgccgggtaa

SEQ ID NO: 82 - DNA, 28 bp, artificial sequence
  Synthetic
cagtcatttggccttggttcagggaggg

SEQ ID NO: 83 - DNA, 25 bp, artificial sequence
  Synthetic
gcaacattcattgttgtcggtgggt

SEQ ID NO: 84 - DNA, 28 bp, artificial sequence
  Synthetic
cagtcatttggcgcctgctggggtggaa

SEQ ID NO: 85 - DNA, 22 bp, artificial sequence
  Synthetic
ggagacgcggccctgttggagt

SEQ ID NO: 86 - DNA, 28 bp, artificial sequence
  Synthetic
cagtcatttggcaagacgggaggaaaga

SEQ ID NO: 87 - DNA, 23 bp, artificial sequence
  Synthetic
cctgaagctcagagggctctgat

SEQ ID NO: 88 - DNA, 19 bp, artificial sequence
  Synthetic
ttgcagctgcctgggagtg

SEQ ID NO: 89 - DNA, 25 bp, artificial sequence
  Synthetic
gctaatccttgctacctgggtgaga

SEQ ID NO: 90 - DNA, 28 bp, artificial sequence
  Synthetic
cagtcatttggcctggccctctctgccc

SEQ ID NO: 91 - DNA, 28 bp, artificial sequence
  Synthetic
cagtcatttggcggcggagggaagtagg

SEQ ID NO: 92 - DNA, 22 bp, artificial sequence
  Synthetic
acaggtgaggttcttgggagcc

SEQ ID NO: 93 - DNA, 22 bp, artificial sequence
  Synthetic
atcatgatgggctcctcggtgt

SEQ ID NO: 94 - DNA, 28 bp, artificial sequence
  Synthetic
cagtcatttggctaaggcacgcggtgaa

SEQ ID NO: 95 - DNA, 23 bp, artificial sequence
  Synthetic
aggaagccctggaggggctggag

SEQ ID NO: 96 - DNA, 22 bp, artificial sequence
  Synthetic
cacatgaaaaggggagagggca

SEQ ID NO: 97 - DNA, 24 bp, artificial sequence
  Synthetic
gctggagaagactggagagggtat

SEQ ID NO: 98 - DNA, 28 bp, artificial sequence
  Synthetic
cccaggctggagtgtagtggcgtgatct

SEQ ID NO: 99 - DNA, 22 bp, artificial sequence
  Synthetic
aactggggcgggaagggggaag

SEQ ID NO: 100 - DNA, 28 bp, artificial sequence
  Synthetic
cagtcatttggcagcagaagcagggagg

SEQ ID NO: 101 - DNA, 23 bp, artificial sequence
  Synthetic
tggtgcggagagggcccacagtg

SEQ ID NO: 102 - DNA, 28 bp, artificial sequence
  Synthetic
cagtcatttggctggaggagaaggaagg

SEQ ID NO: 103 - DNA, 19 bp, artificial sequence
  Synthetic
catagcccggtcgctggta

SEQ ID NO: 104 - DNA, 23 bp, artificial sequence
  Synthetic
ccagttaccgcttccgctaccgc

SEQ ID NO: 105 - DNA, 22 bp, artificial sequence
  Synthetic
tgagacctctgggttctgagct

SEQ ID NO: 106 - DNA, 28 bp, artificial sequence
  Synthetic
cagtcatttggctgtctgcccgcatgcc

SEQ ID NO: 107 - DNA, 22 bp, artificial sequence
  Synthetic
tcggggatcatcatgtcacgag

SEQ ID NO: 108 - DNA, 25 bp, artificial sequence
  Synthetic
gcatcaacagacattaattgggcgc

SEQ ID NO: 109 - DNA, 20 bp, artificial sequence
  Synthetic
accaggaggctgaggcccct

SEQ ID NO: 110 - DNA, 28 bp, artificial sequence
  Synthetic
cagtcatttggcactccagccccacagc

SEQ ID NO: 111 - DNA, 18 bp, artificial sequence
  Synthetic
tttccggctcgcgtgggt

SEQ ID NO: 112 - DNA, 23 bp, artificial sequence
  Synthetic
tgagcgcctcgacgacagagccg

SEQ ID NO: 113 - DNA, 28 bp, artificial sequence
  Synthetic
cagtcatttggcatgcacctgggcaagg

SEQ ID NO: 114 - DNA, 28 bp, artificial sequence
  Synthetic
cagtcatttgggacagcaggcacagaca

SEQ ID NO: 115 - DNA, 28 bp, artificial sequence
  Synthetic
cagtcatttggctgggtctttgcgggcg

SEQ ID NO: 116 - DNA, 28 bp, artificial sequence
  Synthetic
cagtcatttggcgctgactcctagtcca

SEQ ID NO: 117 - DNA, 22 bp, artificial sequence
  Synthetic
ccgcactgtgggtacttgctgc

SEQ ID NO: 118 - DNA, 22 bp, artificial sequence
  Synthetic
actgctgagctagcacttcccg

SEQ ID NO: 119 - DNA, 28 bp, artificial sequence
  Synthetic
cagtcatttgggcccagtgttcagacta

SEQ ID NO: 120 - DNA, 28 bp, artificial sequence
  Synthetic
cagtcatttggcctagactgaagctcct

SEQ ID NO: 121 - DNA, 28 bp, artificial sequence
  Synthetic
cagtcatttgggtcgaggagctcacagt

SEQ ID NO: 122 - DNA, 28 bp, artificial sequence
  Synthetic
cagtcatttggctggagagaaaggcagt

SEQ ID NO: 123 - DNA, 28 bp, artificial sequence
  Synthetic
cagtcatttggcaaggagctcacagtct

SEQ ID NO: 124 - DNA, 28 bp, artificial sequence
  Synthetic
cagtcatttgggtctcacacagaaatcg

SEQ ID NO: 125 - DNA, 28 bp, artificial sequence
  Synthetic
cagtcatttggctaaggtgcatctagtg

SEQ ID NO: 126 - DNA, 28 bp, artificial sequence
  Synthetic
cagtcatttggcaacatagaggaaattc

SEQ ID NO: 127 - DNA, 28 bp, artificial sequence
  Synthetic
cagtcatttggcgaatgttgctcggtga

SEQ ID NO: 128 - DNA, 28 bp, artificial sequence
  Synthetic
cagtcatttggcgaagttgttcgtggtg

SEQ ID NO: 129 - DNA, 28 bp, artificial sequence
  Synthetic
cagtcatttggcaaacaaacatggtgca

SEQ ID NO: 130 - DNA, 28 bp, artificial sequence
  Synthetic
cagtcatttgggtttgttcgttcggctc

SEQ ID NO: 131 - DNA, 28 bp, artificial sequence
  Synthetic
cagtcatttggctaggttatccgtgttg

SEQ ID NO: 132 - DNA, 28 bp, artificial sequence
  Synthetic
cagtcatttggctgagaactgaattcca

SEQ ID NO: 133 - DNA, 28 bp, artificial sequence
  Synthetic
cagtcatttggctgagaactgaattcca

SEQ ID NO: 134 - DNA, 28 bp, artificial sequence
  Synthetic
cagtcatttggcagaggtagtaggttgc

SEQ ID NO: 135 - DNA, 23 bp, artificial sequence
  Synthetic
ccgttttttttttttggagatgg

SEQ ID NO: 136 - DNA, 18 bp, artificial sequence
  Synthetic
aaaaccgatagtgagtcg

SEQ ID NO: 137 - DNA, 23 bp, artificial sequence
  Synthetic
ccgttttttttttttcaaaacat

SEQ ID NO: 138 - DNA, 23 bp, artificial sequence
  Synthetic
ccgttttttttttttagttctcc

SEQ ID NO: 139 - DNA, 23 bp, artificial sequence
  Synthetic
ccgttttttttttttgagctaca

SEQ ID NO: 140 - DNA, 18 bp, artificial sequence
  Synthetic
aaaaccgatagtgagtcg

SEQ ID NO: 141 - DNA, 18 bp, artificial sequence
  Synthetic
aaaaccgatagtgagtcg

SEQ ID NO: 142 - DNA, 18 bp, artificial sequence
  Synthetic
aaaaccgatagtgagtcg

SEQ ID NO: 143 - DNA, 23 bp, artificial sequence
  Synthetic
ccgttttttttttttgtacccct

SEQ ID NO: 144 - DNA, 23 bp, artificial sequence
  Synthetic
ccgttttttttttttgctgggtg

SEQ ID NO: 145 - DNA, 18 bp, artificial sequence
  Synthetic
aaaaccgatagtgagtcg

SEQ ID NO: 146 - DNA, 23 bp, artificial sequence
  Synthetic
ccgttttttttttttacaaagtt

SEQ ID NO: 147 - DNA, 23 bp, artificial sequence
  Synthetic
ccgttttttttttttagggattc

SEQ ID NO: 148 - DNA, 22 bp, artificial sequence
  Synthetic
cgttttttttttttccatcatt

SEQ ID NO: 149 - DNA, 22 bp, artificial sequence
  Synthetic
cgttttttttttttggggaccc

SEQ ID NO: 150 - DNA, 18 bp, artificial sequence
  Synthetic
aaaaccgatagtgagtcg

SEQ ID NO: 151 - DNA, 24 bp, artificial sequence
  Synthetic
tccgttttttttttttaccaggtt

SEQ ID NO: 152 - DNA, 18 bp, artificial sequence
  Synthetic
aaaaccgatagtgagtcg

SEQ ID NO: 153 - DNA, 23 bp, artificial sequence
  Synthetic
ccgttttttttttttctcccttc

SEQ ID NO: 154 - DNA, 18 bp, artificial sequence
  Synthetic
aaaaccgatagtgagtcg

SEQ ID NO: 155 - DNA, 18 bp, artificial sequence
  Synthetic
aaaaccgatagtgagtcg

SEQ ID NO: 156 - DNA, 18 bp, artificial sequence
  Synthetic
aaaaccgatagtgagtcg

SEQ ID NO: 157 - DNA, 23 bp, artificial sequence
  Synthetic
ccgttttttttttttacggaagg

SEQ ID NO: 158 - DNA, 23 bp, artificial sequence
  Synthetic
ccgttttttttttttaccaacgg

SEQ ID NO: 159 - DNA, 18 bp, artificial sequence
  Synthetic
aaaaccgatagtgagtcg

SEQ ID NO: 160 - DNA, 18 bp, artificial sequence
  Synthetic
aaaaccgatagtgagtcg

SEQ ID NO: 161 - DNA, 23 bp, artificial sequence
  Synthetic
ccgttttttttttttggcattca

SEQ ID NO: 162 - DNA, 18 bp, artificial sequence
  Synthetic
aaaaccgatagtgagtcg

SEQ ID NO: 163 - DNA, 18 bp, artificial sequence
  Synthetic
aaaaccgatagtgagtcg

SEQ ID NO: 164 - DNA, 18 bp, artificial sequence
  Synthetic
aaaaccgatagtgagtcg

SEQ ID NO: 165 - DNA, 18 bp, artificial sequence
  Synthetic
aaaaccgatagtgagtcg

SEQ ID NO: 166 - DNA, 18 bp, artificial sequence
  Synthetic
aaaaccgatagtgagtcg

SEQ ID NO: 167 - DNA, 23 bp, artificial sequence
  Synthetic
ccgttttttttttttgggagaac

SEQ ID NO: 168 - DNA, 18 bp, artificial sequence
  Synthetic
aaaaccgatagtgagtcg

SEQ ID NO: 169 - DNA, 23 bp, artificial sequence
  Synthetic
ccgttttttttttttcatcacct

SEQ ID NO: 170 - DNA, 18 bp, artificial sequence
  Synthetic
aaaaccgatagtgagtcg

SEQ ID NO: 171 - DNA, 18 bp, artificial sequence
  Synthetic
aaaaccgatagtgagtcg

SEQ ID NO: 172 - DNA, 18 bp, artificial sequence
  Synthetic
aaaaccgatagtgagtcg

SEQ ID NO: 173 - DNA, 23 bp, artificial sequence
  Synthetic
ccgttttttttttttagaggcag

SEQ ID NO: 174 - DNA, 18 bp, artificial sequence
  Synthetic
aaaaccgatagtgagtcg

SEQ ID NO: 175 - DNA, 18 bp, artificial sequence
  Synthetic
aaaaccgatagtgagtcg

SEQ ID NO: 176 - DNA, 18 bp, artificial sequence
  Synthetic
aaaaccgatagtgagtcg

SEQ ID NO: 177 - DNA, 23 bp, artificial sequence
  Synthetic
ccgttttttttttttgctgaggc

SEQ ID NO: 178 - DNA, 18 bp, artificial sequence
  Synthetic
aaaaccgatagtgagtcg

SEQ ID NO: 179 - DNA, 18 bp, artificial sequence
  Synthetic
aaaaccgatagtgagtcg

SEQ ID NO: 180 - DNA, 22 bp, artificial sequence
  Synthetic
cgttttttttttttcagaatcc

SEQ ID NO: 181 - DNA, 23 bp, artificial sequence
  Synthetic
ccgttttttttttttactgcctg

SEQ ID NO: 182 - DNA, 23 bp, artificial sequence
  Synthetic
ccgttttttttttttcatctcgc

SEQ ID NO: 183 - DNA, 22 bp, artificial sequence
  Synthetic
cgttttttttttttgagccctg

SEQ ID NO: 184 - DNA, 18 bp, artificial sequence
  Synthetic
aaaaccgatagtgagtcg

SEQ ID NO: 185 - DNA, 18 bp, artificial sequence
  Synthetic
aaaaccgatagtgagtcg

SEQ ID NO: 186 - DNA, 23 bp, artificial sequence
  Synthetic
ccgttttttttttttgaacaggt

SEQ ID NO: 187 - DNA, 23 bp, artificial sequence
  Synthetic
ccgttttttttttttcctcaagg

SEQ ID NO: 188 - DNA, 23 bp, artificial sequence
  Synthetic
ccgttttttttttttactagact

SEQ ID NO: 189 - DNA, 22 bp, artificial sequence
  Synthetic
cgttttttttttttcaggaact

SEQ ID NO: 190 - DNA, 23 bp, artificial sequence
  Synthetic
ccgttttttttttttctcaatag

SEQ ID NO: 191 - DNA, 23 bp, artificial sequence
  Synthetic
ccgttttttttttttacgggtgc

SEQ ID NO: 192 - DNA, 23 bp, artificial sequence
  Synthetic
ccgttttttttttttctaactgc

SEQ ID NO: 193 - DNA, 23 bp, artificial sequence
  Synthetic
ccgttttttttttttacgtggaa

SEQ ID NO: 194 - DNA, 23 bp, artificial sequence
  Synthetic
ccgttttttttttttaggggttc

SEQ ID NO: 195 - DNA, 22 bp, artificial sequence
  Synthetic
cgttttttttttttcgaatcca

SEQ ID NO: 196 - DNA, 23 bp, artificial sequence
  Synthetic
ccgttttttttttttaagaagtg

SEQ ID NO: 197 - DNA, 23 bp, artificial sequence
  Synthetic
ccgttttttttttttcacgcgag

SEQ ID NO: 198 - DNA, 23 bp, artificial sequence
  Synthetic
ccgttttttttttttcgaaggca

SEQ ID NO: 199 - DNA, 23 bp, artificial sequence
  Synthetic
ccgttttttttttttagcctatg

SEQ ID NO: 200 - DNA, 23 bp, artificial sequence
  Synthetic
ccgttttttttttttaacccatg

SEQ ID NO: 201 - DNA, 23 bp, artificial sequence
  Synthetic
ccgttttttttttttaactatgc

SEQ ID NO: 202 - DNA, 22 bp, artificial sequence
  Synthetic
gcgagcacagaattaatacgac

(19) United States
(12) Patent Application Publication
(10) Pub. No.: US 2019/0169690 A1
(43) Pub. Date: Jun. 6, 2019

(54) METHODS AND COMPOSITIONS FOR DIAGNOSIS OF ALZHEIMER'S DISEASE

(71) Applicant: ROSETTA GENOMICS LTD., Rehovot (IL)

(72) Inventor: Yaron Goren, Kefar Hess (IL)

(73) Assignee: ROSETTA GENOMICS LTD., REHOVOT (IL)

(21) Appl. No.: 16/218,863

(22) Filed: Dec. 13, 2018

Related U.S. Application Data
(63) Continuation of application No. 15/097,923, filed on Apr. 13, 2016, now abandoned.
Application No. 15/097,923 is a continuation of application No. 14/238,937, filed on Feb. 14, 2014, now abandoned.
(60) Provisional application No. 61/523,868, filed on Aug. 16, 2011.

(51) Int. Cl. C12Q 1/6883

(52) U.S. Cl. CPC C12Q 1/6883; C12Q 2600/118; C12Q 2600/158

(57) ABSTRACT

The invention provides a method of diagnosing Alzheimer's disease (AD) in a subject by determining the level of expression of one or more miRNAs molecules associated with AD, as well as various nucleic acid molecules relating thereto or derived thereof.

### FIELD OF THE INVENTION

The invention relates in general to microRNA molecules associated with diagnosis of Alzheimer's disease, as well as various nucleic acid molecules relating thereto or derived thereof.

### BACKGROUND OF THE INVENTION

Alzheimer's disease (AD) is a dementing illness characterized by progressive neuronal degeneration, gliosis, and the accumulation of intracellular inclusions (neurofibrillary tangles) and extracellular deposits of amyloid (senile plaques) formed around a core of aggregated amyloid β₄₂ peptide in discrete regions of the basal forebrain, hippocampus, and association cortices. The etiology of sporadic AD is likely multifactorial, with carriage of the apolipoprotein E ε4 (APOE4) allele constituting a strong risk factor for the development of this condition.

AD is an impending healthcare crisis brought on in part by an aging population. This is evidenced by the fact that half of those over the age of 80 years are afflicted with AD. At present, AD is the fourth leading cause of adult deaths in the US alone, at an annual cost of approximately $100 billion. As the lifespan of the world's population increases, this disease will become an even greater problem.

To compound the difficulties of managing the ever-increasing AD healthcare crisis, the definitive diagnosis of AD ante-mortem has to date been extremely difficult. Ante-mortem presumptive diagnosis of the disease is performed primarily by exclusion of other diseases. Definitive post-mortem diagnosis of Alzheimer's disease has been based on determination of the number of neuritic plaques and tangles in brain tissue using specialized staining techniques. However, such diagnostic methods, besides not being applicable to ante-mortem diagnosis, require extensive staining and microscopic examination of several brain sections. Moreover, the plaques and tangles are not confined to individuals having Alzheimer's disease, but also may occur in the brains of normal, elderly individuals or individuals with other diseases.

The existing diagnostic molecular tests for AD and other forms of dementia can be divided into two groups. The first group is based on analysis of single nucleotide polymorphisms (SNP), which is helpful for predicting a higher risk of a disease but not for diagnostics (Bettens et al, Hum Mol Genet. 2010, 19(R1):R4-R11). The second group uses analysis of proteins involved in AD pathogenesis or brain-specific proteins, like neural thread protein (NTP), bodily fluids (Schipper, Alheimer's & Dementia. 2007. 3:325-332). However, these tests are not sufficiently sensitive and specific. Recently published data have demonstrated high sensitivity of AD detection by measuring concentrations of three protein biomarkers (beta-amyloid protein 1-42, total tau protein, and phosphoryiated Maul SIP protein) in the cerebrospinal fluid (CSF) (Meyer et. al., Arch Neurol. 2010. 67:949-956). The high invasiveness of the CSF collection procedure makes such tests impractical and challenging for everyday clinical use.

In recent years, microRNAs (miRNAs, miRs) have emerged as an important novel class of regulatory RNA, which have a profound impact on a wide array of biological processes.

These small (typically 17-24 nucleotides long) non-coding RNA molecules can modulate protein expression patterns by promoting RNA degradation, inhibiting mRNA translation, and also affecting gene transcription. miRs play pivotal roles in diverse processes such as development and differentiation, control of cell proliferation, stress response and metabolism. The expression of many miRs was found to be altered in numerous types of human cancer, and in some cases strong evidence has been put forward in support of the conjecture that such alterations may play a causative role in tumor progression. There are currently about 1223 known human miRs.

Some miRs, including those that are cell-specific, are enriched in certain cellular compartments, particularly in axons, dendrites and synapses, See, e.g., Schratt et al, Nature. 439:283-289, 2006; Lugli et al, J Neurochem. 106:650-661, 2008; Bicker and Schratt, J Mol Med., 12: 1466-1476, 2008; Expression and concentrations of miRNAs are regulated by various physiological and pathological signals. Changes in expression of some miRNAs were found in neurons of Alzheimer's and other neurodegenerative disease patients (Hebert and De Strooper, Trends Neurosci. 32: 199-206, 2009; Saba et al, PLoS One, 2008; 3:e3652).

Earlier definitive diagnosis of AD, ideally prior to clinical manifestations of the disease, would facilitate earlier and potentially more effective treatment of patients afflicted with AD. Thus, there is an unmet need for a more definitive and reliable method for making a diagnosis of AD in a living subject. In particular, simple tests for AD diagnosis that can be performed on readily-accessible biological fluids are needed.

### SUMMARY OF THE INVENTION

The present invention is based in part on the discovery of a panel of miRs whose levels are increased or decreased in the circulation of AD patients.

Circulating nucleic acids in body fluids offer unique opportunities for early diagnosis of the risk of AD. The present invention provides specific nucleic acid sequences for use in the identification, early detection and diagnosis of AD. The nucleic acid sequences can also be used as prognostic markers for prognostic evaluation of a subject based on their expression pattern in a biological sample. The invention further provides a method of minimally-invasive early detection or predisposition of AD.

The invention further provides a method of diagnosing AD in a subject, the method comprising: obtaining a biological sample from a subject; determining an expression profile in said sample of a nucleic acid sequence selected from the group consisting of SEQ ID NOS: 1-67; a fragment thereof or a sequence having at least about 80% identity thereto; and comparing said expression profile to a reference expression profile wherein a difference in the level of expression profile in at least one or more nucleic acid sequence in said biological sample compared to said reference expression profile is diagnostic for AD.

According to some embodiments, relatively high expression levels of a nucleic acid sequence selected from the group consisting of SEQ ID NOS: 1-51; a fragment thereof and a sequence having at least about 80% identity thereto is diagnostic for AD.

According to other embodiments, relatively low expression levels of a nucleic acid sequence selected from the group consisting of SEQ ID NOS: 52-67; a fragment thereof and a sequence having at least about 80% identity thereto is diagnostic for AD.

According to some embodiments, said method further comprising managing subject treatment based on the AD status.

According to some embodiments, said biological sample is selected from the group consisting of bodily fluid, a cell line and a tissue sample. According to one embodiment, the bodily fluid sample is a serum sample. According to another embodiment, said bodily fluid sample is a blood sample.

According to some embodiments, the method comprises determining the expression of at least two nucleic acid sequences. According to some embodiments the method further comprising combining one or more expression ratios. According to some embodiments, the expression levels are determined by a method selected from the group consisting of nucleic acid hybridization, nucleic acid amplification, and a combination thereof. According to some embodiments, the nucleic acid amplification method is real-time PCR (RT-PCR). According to one embodiment, said real-time PCR is quantitative real-time PCR (qRT-PCR).

According to some embodiments, the RT-PCR method comprises forward and reverse primers. According to other embodiments, the forward primer comprises a sequence selected from the group consisting of SEQ ID NOS: 68-134; a fragment thereof and a sequence having at least about 80% identity thereto. According to some embodiments, the real-time PCR method further comprises hybridization with a probe.

According to some embodiments, the probe comprises a nucleic acid sequence that is complementary to a sequence selected from the group consisting of any one of SEQ ID NOS: 1-67; a fragment thereof and sequences at least about 80% identical thereto.

According to other embodiments, the probe comprises a sequence selected from the group consisting of any one of SEQ ID NOS: 135-201; a fragment thereof and a sequence having at least about 80% identity thereto.

The invention further provides a kit for assessing AD in a subject; said kit comprises a probe comprising a nucleic acid sequence that is complementary to a sequence selected from the group consisting of any one of SEQ ID NOS: 1-67; a fragment thereof and sequences having at least about 80% identity thereto. According to some embodiments, said probe comprising a nucleic acid sequence selected from the group consisting of SEQ ID NOS: 135-201; a fragment thereof and sequences having at least about 80% identity thereto. According to other embodiments, the kit further comprises a forward primer comprising a sequence selected from the group consisting of SEQ ID NOS: 68-134; a fragment thereof and sequences having at least about 80% identity thereto. According to other embodiments, the kit further comprises a reverse primer comprising SEQ ID NO: 202, a fragment thereof and sequences having at least about 80% identity thereto.

These and other embodiments of the present invention will become apparent in conjunction with the figures, description and claims that follow.

### BRIEF DESCRIPTION OF THE DRAWINGS

FIGS. 1A-1D are boxplots presentations comparing distributions of the expression (Y axis) of exemplified upregulated statistically significant microRNAs: hsa-miR-1296 (SEQ ID NO: 1) (1A), hsa-miR-424* (SEQ ID NO: 2) (1B), hsa-miR-424 (SEQ ID NO: 3) (1C) and hsa-miR-629 (SEQ ID NO: 4) (1D), in serum samples obtained from AD group (I) or healthy subjects (II). The results are based on Real time PCR, and a higher normalized signal indicates higher amounts of microRNA present in the samples.

Boxplots show the median (horizontal line), 25 to 75 percentile (box), extent of data up to 1.5 times the interquartile range (“whiskers”), and outliers (crosses).

FIGS. 2A-2D are boxplots presentations comparing distributions of the expression (Y axis) of exemplified upregulated statistically significant microRNAs: hsa-miR-143 (SEQ ID NO: 5) (2A), MID-16758 (SEQ ID NO: 6) (2B), MID-18395 (SEQ ID NO: 7) (2C) and MID-16748 (SEQ ID NO: 8) (2D), in serum samples obtained from AD group (I) or healthy subjects (II). The results are based on Real time PCR, and a higher normalized signal indicates higher amounts of microRNA present in the samples.

Boxplots show the median (horizontal line), 25 to 75 percentile (box), extent of data up to 1.5 times the interquartile range (“whiskers”), and outliers (crosses).

FIGS. 3A-3D are boxplots presentations comparing distributions of the expression (Y axis) of exemplified upregulated statistically significant microRNAs: hsa-miR-361-5p (SEQ ID NO: 9) (3A), hsa-miR-197 (SEQ ID NO: 10) (3B), MID-16582—(SEQ ID NO: 11) (3C) and hsa-miR-148a (SEQ ID NO: 12) (3D), in serum samples obtained from AD group (I) or healthy subjects (II). The results are based on Real time PCR, and a higher normalized signal indicates higher amounts of microRNA present in the samples.

Boxplots show the median (horizontal line), 25 to 75 percentile (box), extent of data up to 1.5 times the interquartile range (“whiskers”), and outliers (crosses).

FIG. 4A-4D are boxplots presentations comparing distributions of the expression (Y axis) of exemplified up/downregulated statistically significant microRNAs: hsa-miR-145 (SEQ ID NO: 13) (4A), hsa-miR-199a-5p (SEQ ID NO: 52) (4B), hsa-miR-151-3p (SEQ ID NO: 53) (4C) and hsa-miR-151-5p (SEQ ID NO: 54) (4D), in serum samples obtained from AD group (I) or healthy subjects (II). The results are based on Real time PCR, and a higher normalized signal indicates higher amounts of microRNA present in the samples.

Boxplots show the median (horizontal line), 25 to 75 percentile (box), extent of data up to 1.5 times the interquartile range (“whiskers”), and outliers (crosses).

### DETAILED DESCRIPTION OF THE INVENTION

The invention is based in part on the discovery that specific biomarker sequences (SEQ ID NOS: 1-67) can be used for the identification, early detection, diagnosis and prognosis of AD.

Biomarkers have the potential to revolutionize diagnosis and treatment of various medical conditions. Ideally, biomarkers should be sampled in a minimal-invasive way. Therefore the challenge of diverse biomedical research fields has been to identify biomarkers in body fluids, such as serum or blood. In recent years it has become clear that both cell-free DNA and mRNA are present in serum, as well as in other body fluids, and represent potential biomarkers. However, monitoring the typically small amounts of these nucleic acids in body fluids requires sensitive detection methods, which are not currently clinically applicable.

The present invention provides a sensitive, specific and accurate method which can be used for conducting in a minimally-invasive early detection, diagnosis and prognosis of AD. The methods of the present invention have high sensitivity and specificity.

Surprisingly, the above method allows simple minimally-invasive test, for easy detection of AD at a very early stage with higher reliability and effectiveness, saving time, material and operating steps, as well as saving cost and fine chemicals difficult to obtain.

Furthermore, the method according to the invention combines the advantages of easy sample collection and the option of diagnosing AD at an early stage. Being a minimally-invasive method, in which e.g. delivering a sample of serum, the method has a good potential to achieve high acceptance among subjects, which subjects can be humans or animals, for example. Therefore, the method can be used in routine tests, but also in prophylactic medical examinations.

Also, the present invention provides methods for determining a treatment plan. Once the health care provider knows to which disease class the sample, and therefore, the individual belongs, the health care provider can determine an adequate treatment plan for the individual.

### Definitions

Before the present compositions and methods are disclosed and described, it is to be understood that the terminology used herein is for the purpose of describing particular embodiments only and is not intended to be limiting. It must be noted that, as used in the specification and the appended claims, the singular forms “a,” “an” and “the” include plural referents unless the context clearly dictates otherwise.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9 and 7.0 are explicitly contemplated.

About

The term “about” or “approximately” means within a statistically meaningful range of a value. Such a range can be within an order of magnitude, preferably within 50%, more preferably within 20%, still more preferably within 10%, and even more preferably within 5% of a given value or range. The allowable variation encompassed by the term “about” or “approximately” depends on the particular system under study, and can be readily appreciated by one of ordinary skill in the art.

Antisense

The term “antisense,” as used herein, refers to nucleotide sequences which are complementary to a specific DNA or RNA sequence. The term “antisense strand” is used in reference to a nucleic acid strand that is complementary to the “sense” strand. Antisense molecules may be produced by any method, including synthesis by ligating the gene(s) of interest in a reverse orientation to a viral promoter which permits the synthesis of a complementary strand. Once introduced into a cell, this transcribed strand combines with natural sequences produced by the cell to form duplexes. These duplexes then block either the further transcription or translation. In this manner, mutant phenotypes may be generated.

Attached

“Attached” or “immobilized” as used herein refer to a probe and a solid support and may mean that the binding between the probe and the solid support is sufficient to be stable under conditions of binding, washing, analysis, and removal. The binding may be covalent or non-covalent. Covalent bonds may be formed directly between the probe and the solid support or may be formed by a cross linker or by inclusion of a specific reactive group on either the solid support or the probe, or both. Non-covalent binding may be one or more of electrostatic, hydrophilic, and hydrophobic interactions. Included in non-covalent binding is the covalent attachment of a molecule, such as streptavidin, to the support and the non-covalent binding of a biotinylated probe to the streptavidin. Immobilization may also involve a combination of covalent and non-covalent interactions.

Associated With

The term “associated with” is used to encompass any correlation, cooccurrence and any cause-and-effect relationship.

Biological Sample

“Biological sample” as used herein means a sample of biological tissue or fluid that comprises nucleic acids. Such samples include, but are not limited to, tissue or fluid isolated from subjects. Biological samples may also include sections of tissues such as biopsy and autopsy samples, FFPE samples, frozen sections taken for histological purposes, blood, plasma, serum, sputum, stool, tears, mucus, hair, and skin. Biological samples also include explants and primary and/or transformed cell cultures derived from animal or patient tissues.

Biological samples may also be blood, a blood fraction, urine, effusions, ascitic fluid, saliva, cerebrospinal fluid, cervical secretions, vaginal secretions, endometrial secretions, gastrointestinal secretions, bronchial secretions, sputum, cell line, tissue sample, or secretions from the breast. A biological sample may be provided by removing a sample of cells from an animal, but can also be accomplished by using previously isolated cells (e.g., isolated by another person, at another time, and/or for another purpose), or by performing the methods described herein in vivo. Archival tissues, such as those having treatment or outcome history, may also be used.

Classification

“Classification” as used herein refers to a procedure and/or algorithm in which individual items are placed into groups or classes based on quantitative information on one or more characteristics inherent in the items (referred to as traits, variables, characters, features, etc) and based on a statistical model and/or a training set of previously labeled items. According to one embodiment, classification means determination of Alzheimer's disease.

Complement

“Complement” or “complementary” as used herein means Watson-Crick (e.g., A-T/U and C-G) or Hoogsteen base pairing between nucleotides or nucleotide analogs of nucleic acid molecules. A full complement or fully complementary may mean 100% complementary base pairing between nucleotides or nucleotide analogs of nucleic acid molecules. In some embodiments, the complementary sequence has a reverse orientation (5′-3′).

Control Level

The term “a control level” as used herein encompasses predetermined standards (e.g., a value in a reference) as well as levels determined experimentally in similarly processed samples from control subjects (e.g., age-matched healthy subjects, placebo treated patients, etc.).

Correlated

The terms “correlated” and “correlating,” as used herein in reference to the use of diagnostic and prognostic miRNA levels associated with AD or related disorders, refers to comparing the presence or quantity of the miRNA levels in a subject to its presence or quantity in subjects known to suffer from, or known to be at risk of AD (e.g., due to advanced age or other known risk factors); or in subjects known to be free of a given condition, i.e. “normal subjects” or “control subjects”. For example, a level of one or more miRNAs in a biological sample can be compared to a miRNA level for each of the specific miRNAs tested and determined to be correlated with AD. The sample's one or more miRNA levels is said to have been correlated with a diagnosis; that is, the skilled artisan can use the miRNA level(s) to determine whether the subject suffers from AD, or may potentially develop AD, and respond accordingly. Alternatively, the sample's miRNA level(s) can be compared to control miRNA level(s) known to be associated with a good outcome (e.g., the absence of AD), such as an average level found in a population of normal subjects.

In certain embodiments, a diagnostic or prognostic miRNA level is correlated to AD by merely its presence or absence. In other embodiments, a threshold level of a diagnostic or prognostic miRNA level can be established, and the level of the miRNA in a subject sample can simply be compared to the threshold level.

C_{T}

C_{T} signals represent the first cycle of PCR where amplification crosses a threshold (cycle threshold) of fluorescence. Accordingly, low values of C_{T} represent high abundance or expression levels of the microRNA.

In some embodiments the PCR C_{T} signal is normalized such that the normalized C_{T} remains inversed from the expression level. In other embodiments the PCR C_{T} signal may be normalized and then inverted such that low normalized-inverted C_{T} represents low abundance or expression levels of the microRNA.

Detection

“Detection” means detecting the presence of a component in a sample. Detection also means detecting the absence of a component. Detection also means measuring the level of a component, either quantitatively or qualitatively.

Determining the Prognosis

The phrase “determining the prognosis” as used herein refers to methods by which the skilled artisan can predict the course or outcome of a condition in a subject. The term “prognosis” does not refer to the ability to predict the course or outcome of a condition with 100% accuracy, or even that a given course or outcome is predictably more or less likely to occur based on the presence, absence or levels of a biomarker. Instead, the skilled artisan will understand that the term “prognosis” refers to an increased probability that a certain course or outcome will occur; that is, that a course or outcome is more likely to occur in a subject exhibiting a given condition, when compared to those individuals not exhibiting the condition. For example, in individuals not exhibiting the condition (e.g., not expressing the miRNA level(s) or expressing miRNA level(s) at a reduced level), the chance of a given outcome (e.g., suffering from AD) may be very low (e.g., <1%), or even absent. In contrast, in individuals exhibiting the condition (e.g., expressing the miRNA level(s) or expressing miRNA level(s) at a level greatly increased over a control level), the chance of a given outcome (e.g., suffering from AD) may be high. In certain embodiments, a prognosis is about a 5% chance of a given expected outcome, about a 7% chance, about a 10% chance, about a 12% chance, about a 15% chance, about a 20% chance, about a 25% chance, about a 30% chance, about a 40% chance, about a 50% chance, about a 60% chance, about a 75% chance, about a 90% chance, or about a 95% chance.

The skilled artisan will understand that associating a prognostic indicator with a predisposition to an adverse outcome is a statistical analysis. For example, miRNA level(s) (e.g., quantity of one or more miRNAs in a sample) of greater than a control level in some embodiments can signal that a subject is more likely to suffer from AD than subjects with a level less than or equal to the control level, as determined by a level of statistical significance. Additionally, a change in miRNA level(s) from baseline levels can be reflective of subject prognosis, and the degree of change in marker level can be related to the severity of adverse events. Statistical significance is often determined by comparing two or more populations, and determining a confidence interval and/or a p value.

Diagnosis

The terms “diagnosing” and “diagnosis” as used herein refer to methods by which the skilled artisan can estimate and even determine whether or not a subject is suffering from a given disease or condition. The skilled artisan often makes a diagnosis on the basis of one or more diagnostic indicators, such as for example a biomarker, the amount (including presence or absence) of which is indicative of the presence, severity, or absence of the condition.

Along with diagnosis, clinical disease prognosis is also an area of great concern and interest. It is important to know the stage and rapidity of advancement of the AD in order to plan the most effective therapy. If a more accurate prognosis can be made, appropriate therapy, and in some instances less severe therapy for the patient can be chosen. Measurement of miRNA levels disclosed herein can be useful in order to categorize subjects according to advancement of AD who will benefit from particular therapies and differentiate from other subjects where alternative or additional therapies can be more appropriate.

As such, “making a diagnosis” or “diagnosing”, as used herein, is further inclusive of making a prognosis, which can provide for predicting a clinical outcome (with or without medical treatment), selecting an appropriate treatment (or whether treatment would be effective), or monitoring a current treatment and potentially changing the treatment, based on the measure of diagnostic miRNA levels. Further, in some embodiments of the presently-disclosed subject matter, multiple determinations of amounts of one or more miRNAs over time can be made to facilitate diagnosis and/or prognosis. A temporal change in one or more miRNA levels (i.e., miRNA amounts in a biological sample) can be used to predict a clinical outcome, monitor the progression of the AD, and/or efficacy of administered AD therapies. In such an embodiment for example, one could observe a decrease in the amount of particular miRNAs (as disclosed in greater detail in the Examples) in a biological sample over time during the course of a therapy, thereby indicating effectiveness of treatment.

In some embodiments, a first time point can be selected prior to initiation of a prophylaxis or treatment and a second time point can be selected at some time after initiation of the prophylaxis or treatment. miRNA levels can be measured in each of the samples taken from different time points and qualitative and/or quantitative differences noted. A change in the amounts of one or more of the measured miRNA levels from the first and second samples can be correlated with prognosis, used to determine treatment efficacy, and/or used to determine progression of the disease in the subject.

Differential Expression

“Differential expression” means qualitative or quantitative differences in the temporal and/or cellular gene expression patterns within and among cells and tissue. Thus, a differentially expressed gene may qualitatively have its expression altered, including an activation or inactivation, in, e.g., normal versus disease tissue. Genes may be turned on or turned off in a particular state, relative to another state thus permitting comparison of two or more states. A qualitatively regulated gene may exhibit an expression pattern within a state or cell type which may be detectable by standard techniques. Some genes may be expressed in one state or cell type, but not in both. Alternatively, the difference in expression may be quantitative, e.g., in that expression is modulated, either up-regulated, resulting in an increased amount of transcript, or down-regulated, resulting in a decreased amount of transcript. The degree to which expression differs need only be large enough to quantify via standard characterization techniques such as expression arrays, quantitative reverse transcriptase PCR, northern analysis, real-time PCR, in situ hybridization and RNase protection.

Expression Profile

The term “expression profile” is used broadly to include a genomic expression profile, e.g., an expression profile of microRNAs. Profiles may be generated by any convenient means for determining a level of a nucleic acid sequence e.g. quantitative hybridization of microRNA, labeled microRNA, amplified microRNA, cDNA, etc., quantitative PCR, ELISA for quantitation, and the like, and allow the analysis of differential gene expression between two samples. A subject or patient tumor sample, e.g., cells or collections thereof, e.g., tissues, is assayed. Samples are collected by any convenient method, as known in the art. Nucleic acid sequences of interest are nucleic acid sequences that are found to be predictive, including the nucleic acid sequences provided above, where the expression profile may include expression data for 5, 10, 20, 25, 50, 100 or more of, including all of the listed nucleic acid sequences. According to some embodiments, the term “expression profile” means measuring the abundance of the nucleic acid sequences in the measured samples.

Expression Ratio

“Expression ratio” as used herein refers to relative expression levels of two or more nucleic acids as determined by detecting the relative expression levels of the corresponding nucleic acids in a biological sample.

FDR

When performing multiple statistical tests, for example in comparing the signal between two groups in multiple data features, there is an increasingly high probability of obtaining false positive results, by random differences between the groups that can reach levels that would otherwise be considered as statistically significant. In order to limit the proportion of such false discoveries, statistical significance is defined only for data features in which the differences reached a p-value (by two-sided t-test) below a threshold, which is dependent on the number of tests performed and the distribution of p-values obtained in these tests.

Fragment

“Fragment” is used herein to indicate a non-full length part of a nucleic acid or polypeptide. Thus, a fragment is itself also a nucleic acid or polypeptide, respectively.

Gene

“Gene” as used herein may be a natural (e.g., genomic) or synthetic gene comprising transcriptional and/or translational regulatory sequences and/or a coding region and/or non-translated sequences (e.g., introns, 5′- and 3′-untranslated sequences). The coding region of a gene may be a nucleotide sequence coding for an amino acid sequence or a functional RNA, such as tRNA, rRNA, catalytic RNA, siRNA, miRNA or antisense RNA. A gene may also be an mRNA or cDNA corresponding to the coding regions (e.g., exons and miRNA) optionally comprising 5′- or 3′-untranslated sequences linked thereto. A gene may also be an amplified nucleic acid molecule produced in vitro comprising all or a part of the coding region and/or 5′- or 3′-untranslated sequences linked thereto.

Groove Binder/Minor Groove Binder (MGB)

“Groove binder” and/or “minor groove binder” may be used interchangeably and refer to small molecules that fit into the minor groove of double-stranded DNA, typically in a sequence-specific manner. Minor groove binders may be long, flat molecules that can adopt a crescent-like shape and thus, fit snugly into the minor groove of a double helix, often displacing water. Minor groove binding molecules may typically comprise several aromatic rings connected by bonds with torsional freedom such as furan, benzene, or pyrrole rings. Minor groove binders may be antibiotics such as netropsin, distamycin, berenil, pentamidine and other aromatic diamidines, Hoechst 33258, SN 6999, aureolic anti-tumor drugs such as chromomycin and mithramycin, CC-1065, dihydrocyclopyrroloindole tripeptide (DPI₃), 1,2-dihydro-(3H)-pyrrolo[3,2-e]indole-7-carboxylate (CDPI₃), and related compounds and analogues, including those described in Nucleic Acids in Chemistry and Biology, 2d ed., Blackburn and Gait, eds., Oxford University Press, 1996, and PCT Published Application No. WO 03/078450, the contents of which are incorporated herein by reference. A minor groove binder may be a component of a primer, a probe, a hybridization tag complement, or combinations thereof. Minor groove binders may increase the Tₘ of the primer or a probe to which they are attached, allowing such primers or probes to effectively hybridize at higher temperatures.

Identity

“Identical” or “identity” as used herein in the context of two or more nucleic acids or polypeptide sequences mean that the sequences have a specified percentage of residues that are the same over a specified region. The percentage may be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of the single sequence are included in the denominator but not the numerator of the calculation. When comparing DNA and RNA, thymine (T) and uracil (U) may be considered equivalent. Identity may be performed manually or by using a computer sequence algorithm such as BLAST or BLAST 2.0.

In Situ Detection

“In situ detection” as used herein means the detection of expression or expression levels in the original site hereby meaning in a tissue sample such as biopsy.

Label

“Label” as used herein means a composition detectable by spectroscopic, photochemical, biochemical, immunochemical, chemical, or other physical means. For example, useful labels include ³²P, fluorescent dyes, electron-dense reagents, enzymes (e.g., as commonly used in an ELISA), biotin, digoxigenin, or haptens and other entities which can be made detectable. A label may be incorporated into nucleic acids and proteins at any position.

Nucleic Acid

“Nucleic acid” or “oligonucleotide” or “polynucleotide” as used herein mean at least two nucleotides covalently linked together. The depiction of a single strand also defines the sequence of the complementary strand. Thus, a nucleic acid also encompasses the complementary strand of a depicted single strand. Many variants of a nucleic acid may be used for the same purpose as a given nucleic acid. Thus, a nucleic acid also encompasses substantially identical nucleic acids and complements thereof. A single strand provides a probe that may hybridize to a target sequence under stringent hybridization conditions. Thus, a nucleic acid also encompasses a probe that hybridizes under stringent hybridization conditions.

Nucleic acids may be single stranded or double stranded, or may contain portions of both double stranded and single stranded sequence. The nucleic acid may be DNA, both genomic and cDNA, RNA, or a hybrid, where the nucleic acid may contain combinations of deoxyribo- and ribo-nucleotides, and combinations of bases including uracil, adenine, thymine, cytosine, guanine, inosine, xanthine hypoxanthine, isocytosine and isoguanine. Nucleic acids may be obtained by chemical synthesis methods or by recombinant methods.

A nucleic acid will generally contain phosphodiester bonds, although nucleic acid analogs may be included that may have at least one different linkage, e.g., phosphoramidate, phosphorothioate, phosphorodithioate, or O-methylphosphoroamidite linkages and peptide nucleic acid backbones and linkages. Other analog nucleic acids include those with positive backbones; non-ionic backbones, and non-ribose backbones, including those described in U.S. Pat. Nos. 5,235,033 and 5,034,506, which are incorporated by reference. Nucleic acids containing one or more non-naturally occurring or modified nucleotides are also included within one definition of nucleic acids. The modified nucleotide analog may be located for example at the 5′-end and/or the 3′-end of the nucleic acid molecule. Representative examples of nucleotide analogs may be selected from sugar- or backbone-modified ribonucleotides. It should be noted, however, that also nucleobase-modified ribonucleotides, i.e. ribonucleotides, containing a non-naturally occurring nucleobase instead of a naturally occurring nucleobase such as uridines or cytidines modified at the 5-position, e.g. 5-(2-amino) propyl uridine, 5-bromo uridine; adenosines and guanosines modified at the 8-position, e.g. 8-bromo guanosine; deaza nucleotides, e.g. 7-deaza-adenosine; O- and N-alkylated nucleotides, e.g. N6-methyl adenosine are suitable. The 2′-OH-group may be replaced by a group selected from H, OR, R, halo, SH, SR, NH₂, NHR, NR₂ or CN, wherein R is C₁-C₆ alkyl, alkenyl or alkynyl and halo is F, Cl, Br or I. Modified nucleotides also include nucleotides conjugated with cholesterol through, e.g., a hydroxyprolinol linkage as described in Krutzfeldt et al., Nature 438:685-689 (2005) and Soutschek et al., Nature 432:173-178 (2004), which are incorporated herein by reference. Modifications of the ribose-phosphate backbone may be done for a variety of reasons, e.g., to increase the stability and half-life of such molecules in physiological environments, to enhance diffusion across cell membranes, or as probes on a biochip. The backbone modification may also enhance resistance to degradation, such as in the harsh endocytic environment of cells. The backbone modification may also reduce nucleic acid clearance by hepatocytes, such as in the liver. Mixtures of naturally occurring nucleic acids and analogs may be made; alternatively, mixtures of different nucleic acid analogs, and mixtures of naturally occurring nucleic acids and analogs may be made.

Probe

“Probe” as used herein means an oligonucleotide capable of binding to a target nucleic acid of complementary sequence through one or more types of chemical bonds, usually through complementary base pairing, usually through hydrogen bond formation. Probes may bind target sequences lacking complete complementarity with the probe sequence depending upon the stringency of the hybridization conditions. There may be any number of base pair mismatches which will interfere with hybridization between the target sequence and the single stranded nucleic acids described herein. However, if the number of mutations is so great that no hybridization can occur under even the least stringent of hybridization conditions, the sequence is not a complementary target sequence. A probe may be single stranded or partially single and partially double stranded. The strandedness of the probe is dictated by the structure, composition, and properties of the target sequence. Probes may be directly labeled or indirectly labeled such as with biotin to which a streptavidin complex may later bind.

Reference Expression Profile

As used herein, the phrase “reference expression profile” refers to a criterion expression value to which measured values are compared in order to determine the detection of a subject with AD. The reference may be based on a combine metric score.

Sensitivity

“sensitivity” used herein may mean a statistical measure of how well a binary classification test correctly identifies a condition, for example how frequently it correctly classifies AD. The sensitivity for class A is the proportion of cases that are determined to belong to class “A” by the test out of the cases that are in class “A”, as determined by some absolute or gold standard.

Specificity

“Specificity” used herein may mean a statistical measure of how well a binary classification test correctly identifies a condition, for example how frequently it correctly classifies AD. The specificity for class A is the proportion of cases that are determined to belong to class “not A” by the test out of the cases that are in class “not A”, as determined by some absolute or gold standard.

Standard Sample

A “standard sample” refers to a sample that is representative of a disease-free state, particularly a state in which AD or any other associated condition is absent (i.e. a healthy state). By way of example, the standard sample may be a biological sample, obtained from a healthy subject of similar age as the subject for whom the diagnosis or prognosis is provided. A standard sample may be a composite sample, wherein data obtained from biological samples from several healthy subjects (i.e. control subjects who do not have symptoms of AD) are averaged, thereby creating the composite sample.

Stringent Hybridization Conditions

“Stringent hybridization conditions” as used herein mean conditions under which a first nucleic acid sequence (e.g., probe) will hybridize to a second nucleic acid sequence (e.g., target), such as in a complex mixture of nucleic acids. Stringent conditions are sequence-dependent and will be different in different circumstances. Stringent conditions may be selected to be about 5-10° C. lower than the thermal melting point (Tₘ) for the specific sequence at a defined ionic strength pH. The Tₘ may be the temperature (under defined ionic strength, pH, and nucleic acid concentration) at which 50% of the probes complementary to the target hybridize to the target sequence at equilibrium (as the target sequences are present in excess, at Tₘ, 50% of the probes are occupied at equilibrium).

Stringent conditions may be those in which the salt concentration is less than about 1.0 M sodium ion, such as about 0.01-1.0 M sodium ion concentration (or other salts) at pH 7.0 to 8.3 and the temperature is at least about 30° C. for short probes (e.g., about 10-50 nucleotides) and at least about 60° C. for long probes (e.g., greater than about 50 nucleotides). Stringent conditions may also be achieved with the addition of destabilizing agents such as formamide. For selective or specific hybridization, a positive signal may be at least 2 to 10 times background hybridization. Exemplary stringent hybridization conditions include the following: 50% formamide, 5×SSC, and 1% SDS, incubating at 42° C., or, 5×SSC, 1% SDS, incubating at 65° C., with wash in 0.2×SSC, and 0.1% SDS at 65° C.

Substantially Complementary

“Substantially complementary” as used herein means that a first sequence is at least 60%, 65%, 70%, 75%, 80%, 85%, 90%, 95%, 97%, 98% or 99% identical to the complement of a second sequence over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100 or more nucleotides, or that the two sequences hybridize under stringent hybridization conditions.

Substantially Identical

“Substantially identical” as used herein means that a first and a second sequence are at least 60%, 65%, 70%, 75%, 80%, 85%, 90%, 95%, 97%, 98% or 99% identical over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100 or more nucleotides or amino acids, or with respect to nucleic acids, if the first sequence is substantially complementary to the complement of the second sequence.

Subject

As used herein, the term “subject” refers to a mammal, including both human and other mammals. The methods of the present invention are preferably applied to human subjects.

Target Nucleic Acid

“Target nucleic acid” as used herein means a nucleic acid or variant thereof that may be bound by another nucleic acid. A target nucleic acid may be a DNA sequence. The target nucleic acid may be RNA. The target nucleic acid may comprise a mRNA, tRNA, shRNA, siRNA or Piwi-interacting RNA, or a pri-miRNA, pre-miRNA, miRNA, or anti-miRNA.

The target nucleic acid may comprise a target miRNA binding site or a variant thereof. One or more probes may bind the target nucleic acid. The target binding site may comprise 5-100 or 10-60 nucleotides. The target binding site may comprise a total of 5, 6, 7, 8, 9, 10, 11, 12, 13, 14, 15,16, 17, 18, 19, 20, 21, 22, 23, 24, 25, 26, 27, 28, 29, 30-40, 40-50, 50-60, 61, 62 or 63 nucleotides. The target site sequence may comprise at least 5 nucleotides of the sequence of a target miRNA binding site disclosed in U.S. patent application Ser. Nos. 11/384,049, 11/418,870 or 11/429,720, the contents of which are incorporated herein.

Tissue Sample

As used herein, a tissue sample is tissue obtained from a tissue biopsy using methods well known to those of ordinary skill in the related medical arts. Methods for obtaining the sample from the biopsy include gross apportioning of a mass, microdissection, laser-based microdissection, or other art-known cell-separation methods.

Variant

“Variant” as used herein referring to a nucleic acid means (i) a portion of a referenced nucleotide sequence; (ii) the complement of a referenced nucleotide sequence or portion thereof; (iii) a nucleic acid that is substantially identical to a referenced nucleic acid or the complement thereof; or (iv) a nucleic acid that hybridizes under stringent conditions to the referenced nucleic acid, complement thereof, or a sequence substantially identical thereto.

Wild Type

As used herein, the term “wild type” sequence refers to a coding, a non-coding or an interface sequence which is an allelic form of sequence that performs the natural or normal function for that sequence. Wild type sequences include multiple allelic forms of a cognate sequence, for example, multiple alleles of a wild type sequence may encode silent or conservative changes to the protein sequence that a coding sequence encodes.

The present invention employs miRNA for the identification, classification and diagnosis of Alzheimer's disease.

MicroRNA Processing

A gene coding for a microRNA (miRNA) may be transcribed leading to production of an miRNA precursor known as the pri-miRNA. The pri-miRNA may be part of a polycistronic RNA comprising multiple pri-miRNAs. The pri-miRNA may form a hairpin structure with a stem and loop. The stem may comprise mismatched bases.

The hairpin structure of the pri-miRNA may be recognized by Drosha, which is an RNase III endonuclease. Drosha may recognize terminal loops in the pri-miRNA and cleave approximately two helical turns into the stem to produce a 60-70 nucleotide precursor known as the pre-miRNA. Drosha may cleave the pri-miRNA with a staggered cut typical of RNase III endonucleases yielding a pre-miRNA stem loop with a 5′ phosphate and ˜2 nucleotide 3′ overhang. Approximately one helical turn of the stem (˜10 nucleotides) extending beyond the Drosha cleavage site may be essential for efficient processing. The pre-miRNA may then be actively transported from the nucleus to the cytoplasm by Ran-GTP and the export receptor Ex-portin-5.

The pre-miRNA may be recognized by Dicer, which is also an RNase III endonuclease. Dicer may recognize the double-stranded stem of the pre-miRNA. Dicer may also recognize the 5′ phosphate and 3′ overhang at the base of the stem loop. Dicer may cleave off the terminal loop two helical turns away from the base of the stem loop leaving an additional 5′ phosphate and ˜2 nucleotide 3′ overhang. The resulting siRNA-like duplex, which may comprise mismatches, comprises the mature miRNA and a similar-sized fragment known as the miRNA*. The miRNA and miRNA* may be derived from opposing arms of the pri-miRNA and pre-miRNA. MiRNA* sequences may be found in libraries of cloned miRNAs but typically at lower frequency than the miRNAs.

Although initially present as a double-stranded species with miRNA*, the miRNA may eventually become incorporated as a single-stranded RNA into a ribonucleoprotein complex known as the RNA-induced silencing complex (RISC). Various proteins can form the RISC, which can lead to variability in specificity for miRNA/miRNA* duplexes, binding site of the target gene, activity of miRNA (repression or activation), and which strand of the miRNA/miRNA* duplex is loaded in to the RISC.

When the miRNA strand of the miRNA:miRNA* duplex is loaded into the RISC, the miRNA* may be removed and degraded. The strand of the miRNA:miRNA* duplex that is loaded into the RISC may be the strand whose 5′ end is less tightly paired. In cases where both ends of the miRNA:miRNA* have roughly equivalent 5′ pairing, both miRNA and miRNA* may have gene silencing activity.

The RISC may identify target nucleic acids based on high levels of complementarity between the miRNA and the mRNA, especially by nucleotides 2-7 of the miRNA. Only one case has been reported in animals where the interaction between the miRNA and its target was along the entire length of the miRNA. This was shown for mir-196 and Hox B8 and it was further shown that mir-196 mediates the cleavage of the Hox B8 mRNA (Yekta et al 2004, Science 304-594). Otherwise, such interactions are known only in plants (Bartel & Bartel 2003, Plant Physiol 132-709).

A number of studies have studied the base-pairing requirement between miRNA and its mRNA target for achieving efficient inhibition of translation (reviewed by Bartel 2004, Cell 116-281). In mammalian cells, the first 8 nucleotides of the miRNA may be important (Doench & Sharp 2004 GenesDev 2004-504). However, other parts of the microRNA may also participate in mRNA binding. Moreover, sufficient base pairing at the 3′ can compensate for insufficient pairing at the 5′ (Brennecke et al, 2005 PLoS 3-e85).

Computation studies, analyzing miRNA binding on whole genomes have suggested a specific role for bases 2-7 at the 5′ of the miRNA in target binding but the role of the first nucleotide, found usually to be “A” was also recognized (Lewis et at 2005 Cell 120-15). Similarly, nucleotides 1-7 or 2-8 were used to identify and validate targets by Krek et al (2005, Nat Genet 37-495).

The target sites in the mRNA may be in the 5′ UTR, the 3′ UTR or in the coding region. Interestingly, multiple miRNAs may regulate the same mRNA target by recognizing the same or multiple sites. The presence of multiple miRNA binding sites in most genetically identified targets may indicate that the cooperative action of multiple RISCs provides the most efficient translational inhibition.

miRNAs may direct the RISC to downregulate gene expression by either of two mechanisms: mRNA cleavage or translational repression. The miRNA may specify cleavage of the mRNA if the mRNA has a certain degree of complementarity to the miRNA. When a miRNA guides cleavage, the cut may be between the nucleotides pairing to residues 10 and 11 of the miRNA. Alternatively, the miRNA may repress translation if the miRNA does not have the requisite degree of complementarity to the miRNA. Translational repression may be more prevalent in animals since animals may have a lower degree of complementarity between the miRNA and the binding site.

It should be noted that there may be variability in the 5′ and 3′ ends of any pair of miRNA and miRNA*. This variability may be due to variability in the enzymatic processing of Drosha and Dicer with respect to the site of cleavage. Variability at the 5′ and 3′ ends of miRNA and miRNA* may also be due to mismatches in the stem structures of the pri-miRNA and pre-miRNA. The mismatches of the stem strands may lead to a population of different hairpin structures. Variability in the stem structures may also lead to variability in the products of cleavage by Drosha and Dicer.

Nucleic Acids

Nucleic acids are provided herein. The nucleic acids comprise the sequence of SEQ ID NOS: 1-202 or variants thereof. The variant may be a complement of the referenced nucleotide sequence. The variant may also be a nucleotide sequence that is substantially identical to the referenced nucleotide sequence or the complement thereof. The variant may also be a nucleotide sequence which hybridizes under stringent conditions to the referenced nucleotide sequence, complements thereof, or nucleotide sequences substantially identical thereto.

The nucleic acid may have a length of from 10 to 250 nucleotides. The nucleic acid may have a length of at least 10, 11, 12, 13, 14, 15, 16, 17, 18, 19, 20, 21, 22, 23, 24, 25, 26, 27, 28, 29, 30, 35, 40, 45, 50, 60, 70, 80, 90, 100, 125, 150, 175, 200 or 250 nucleotides. The nucleic acid may be synthesized or expressed in a cell (in vitro or in vivo) using a synthetic gene described herein. The nucleic acid may be synthesized as a single strand molecule and hybridized to a substantially complementary nucleic acid to form a duplex. The nucleic acid may be introduced to a cell, tissue or organ in a single- or double-stranded form or capable of being expressed by a synthetic gene using methods well known to those skilled in the art, including as described in U.S. Pat. No. 6,506,559 which is incorporated by reference.

Nucleic Acid Complexes

The nucleic acid may further comprise one or more of the following: a peptide, a protein, a RNA-DNA hybrid, an antibody, an antibody fragment, a Fab fragment, and an aptamer.

Pri-miRNA

The nucleic acid may comprise a sequence of a pri-miRNA or a variant thereof. The pri-miRNA sequence may comprise from 45-30,000, 50-25,000, 100-20,000, 1,000-1,500 or 80-100 nucleotides. The sequence of the pri-miRNA may comprise a pre-miRNA, miRNA and miRNA*, as set forth herein, and variants thereof. The sequence of the pri-miRNA may comprise the sequence of SEQ ID NOS: 1-67; or variants thereof.

The pri-miRNA may form a hairpin structure. The hairpin may comprise a first and a second nucleic acid sequence that are substantially complimentary. The first and second nucleic acid sequence may be from 37-50 nucleotides. The first and second nucleic acid sequence may be separated by a third sequence of from 8-12 nucleotides. The hairpin structure may have a free energy of less than -25 Kcal/mole, as calculated by the Vienna algorithm, with default parameters as described in Hofacker et al., Monatshefte f. Chemie 125: 167-188 (1994), the contents of which are incorporated herein. The hairpin may comprise a terminal loop of 4-20, 8-12 or 10 nucleotides. The pri-miRNA may comprise at least 19% adenosine nucleotides, at least 16% cytosine nucleotides, at least 23% thymine nucleotides and at least 19% guanine nucleotides.

Pre-miRNA

The nucleic acid may also comprise a sequence of a pre-miRNA or a variant thereof. The pre-miRNA sequence may comprise from 45-90, 60-80 or 60-70 nucleotides. The sequence of the pre-miRNA may comprise a miRNA and a miRNA* as set forth herein. The sequence of the pre-miRNA may also be that of a pri-miRNA excluding from 0-160 nucleotides from the 5′ and 3′ ends of the pri-miRNA. The sequence of the pre-miRNA may comprise the sequence of SEQ ID NOS: 1-67; or variants thereof.

miRNA

The nucleic acid may also comprise a sequence of a miRNA (including miRNA*) or a variant thereof. The miRNA sequence may comprise from 13-33, 18-24 or 21-23 nucleotides. The miRNA may also comprise a total of at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 nucleotides. The sequence of the miRNA may be the first 13-33 nucleotides of the pre-miRNA. The sequence of the miRNA may also be the last 13-33 nucleotides of the pre-miRNA. The sequence of the miRNA may comprise the sequence of SEQ ID NOS: 1-67; or variants thereof.

Anti-miRNA

The nucleic acid may also comprise a sequence of an anti-miRNA capable of blocking the activity of a miRNA or miRNA*, such as by binding to the pri-miRNA, pre-miRNA, miRNA or miRNA* (e.g. antisense or RNA silencing), or by binding to the target binding site. The anti-miRNA may comprise a total of 5-100 or 10-60 nucleotides. The anti-miRNA may also comprise a total of at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 nucleotides. The sequence of the anti-miRNA may comprise (a) at least 5 nucleotides that are substantially identical or complimentary to the 5′ of a miRNA and at least 5-12 nucleotides that are substantially complimentary to the flanking regions of the target site from the 5′ end of the miRNA, or (b) at least 5-12 nucleotides that are substantially identical or complimentary to the 3′ of a miRNA and at least 5 nucleotide that are substantially complimentary to the flanking region of the target site from the 3′ end of the miRNA. The sequence of the anti-miRNA may comprise the compliment of SEQ ID NOS: 1-67; or variants thereof.

Binding Site of Target

The nucleic acid may also comprise a sequence of a target microRNA binding site or a variant thereof. The target site sequence may comprise a total of 5-100 or 10-60 nucleotides. The target site sequence may also comprise a total of at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62 or 63 nucleotides. The target site sequence may comprise at least 5 nucleotides of the sequence of SEQ ID NOS: 1-67.

Probes

A probe is provided herein. A probe may comprise a nucleic acid. The probe may have a length of from 8 to 500, 10 to 100 or 20 to 60 nucleotides. The probe may also have a length of at least 8, 9, 10, 11, 12, 13, 14, 15, 16, 17, 18, 19, 20, 21, 22, 23, 24, 25, 26, 27, 28, 29, 30, 35, 40, 45, 50, 60, 70, 80, 90, 100, 120, 140, 160, 180, 200, 220, 240, 260, 280 or 300 nucleotides. The probe may comprise a nucleic acid of 18-25 nucleotides.

A probe may be capable of binding to a target nucleic acid of complementary sequence through one or more types of chemical bonds, usually through complementary base pairing, usually through hydrogen bond formation. Probes may bind target sequences lacking complete complementarity with the probe sequence depending upon the stringency of the hybridization conditions. A probe may be single stranded or partially single and partially double stranded. The strandedness of the probe is dictated by the structure, composition, and properties of the target sequence. Probes may be directly labeled or indirectly labeled.

Test Probe

The probe may be a test probe. The test probe may comprise a nucleic acid sequence that is complementary to a miRNA, a miRNA*, a pre-miRNA, or a pri-miRNA. The sequence of the test probe may be selected from SEQ ID NOS: 135-201; or variants thereof.

Linker Sequences

The probe may further comprise a linker. The linker may be 10-60 nucleotides in length.

The linker may be 20-27 nucleotides in length. The linker may be of sufficient length to allow the probe to be a total length of 45-60 nucleotides. The linker may not be capable of forming a stable secondary structure, or may not be capable of folding on itself, or may not be capable of folding on a non-linker portion of a nucleic acid contained in the probe. The sequence of the linker may not appear in the genome of the animal from which the probe non-linker nucleic acid is derived.

Reverse Transcription

Target sequences of a cDNA may be generated by reverse transcription of the target RNA. Methods for generating cDNA may be reverse transcribing polyadenylated RNA or alternatively, RNA with a ligated adaptor sequence.

Reverse Transcription using Adaptor Sequence Ligated to RNA

The RNA may be ligated to an adapter sequence prior to reverse transcription. A ligation reaction may be performed by T4 RNA ligase to ligate an adaptor sequence at the 3′ end of the RNA. Reverse transcription (RT) reaction may then be performed using a primer comprising a sequence that is complementary to the 3′ end of the adaptor sequence.

Reverse Transcription using Polyadenylated Sequence Ligated to RNA

Polyadenylated RNA may be used in a reverse transcription (RT) reaction using a poly(T) primer comprising a 5′ adaptor sequence. The poly(T) sequence may comprise 8, 9, 10, 11, 12, 13, or 14 consecutive thymines.

RT-PCR of RNA

The reverse transcript of the RNA may be amplified by real time PCR, using a specific forward primer comprising at least 15 nucleic acids complementary to the target nucleic acid and a 5′ tail sequence; a reverse primer that is complementary to the 3′ end of the adaptor sequence; and a probe comprising at least 8 nucleic acids complementary to the target nucleic acid. The probe may be partially complementary to the 5′ end of the adaptor sequence.

PCR of Target Nucleic Acids

Methods of amplifying target nucleic acids are described herein. The amplification may be by a method comprising PCR. The first cycles of the PCR reaction may have an annealing temp of 56° C., 57° C., 58° C., 59° C., or 60° C. The first cycles may comprise 1-10 cycles. The remaining cycles of the PCR reaction may be 60° C. The remaining cycles may comprise 2-40 cycles. The annealing temperature may cause the PCR to be more sensitive. The PCR may generate longer products that can serve as higher stringency PCR templates.

Forward Primer

The PCR reaction may comprise a forward primer. The forward primer may comprise 15, 16, 17, 18, 19, 20, or 21 nucleotides identical to the target nucleic acid.

The 3′ end of the forward primer may be sensitive to differences in sequence between a target nucleic acid and a sibling nucleic acid.

The forward primer may also comprise a 5′ overhanging tail. The 5′ tail may increase the melting temperature of the forward primer. The sequence of the 5′ tail may comprise a sequence that is non-identical to the genome of the animal from which the target nucleic acid is isolated. The sequence of the 5′ tail may also be synthetic. The 5′ tail may comprise 8, 9, 10, 11, 12, 13, 14, 15, or 16 nucleotides. The forward primer may comprise SEQ ID NOS: 68-134; or variants thereof.

Reverse Primer

The PCR reaction may comprise a reverse primer. The reverse primer may be complementary to a target nucleic acid. The reverse primer may also comprise a sequence complementary to an adaptor sequence. The sequence complementary to an adaptor sequence may comprise SEQ ID NO: 202, or variants thereof.

Biochip

A biochip is also provided. The biochip may comprise a solid substrate comprising an attached probe or plurality of probes described herein. The probes may be capable of hybridizing to a target sequence under stringent hybridization conditions. The probes may be attached at spatially defined locations on the substrate. More than one probe per target sequence may be used, with either overlapping probes or probes to different sections of a particular target sequence. The probes may be capable of hybridizing to target sequences associated with a single disorder appreciated by those in the art. The probes may either be synthesized first, with subsequent attachment to the biochip, or may be directly synthesized on the biochip.

The solid substrate may be a material that may be modified to contain discrete individual sites appropriate for the attachment or association of the probes and is amenable to at least one detection method. Representative examples of substrate materials include glass and modified or functionalized glass, plastics (including acrylics, polystyrene and copolymers of styrene and other materials, polypropylene, polyethylene, polybutylene, polyurethanes, TeflonJ, etc.), polysaccharides, nylon or nitrocellulose, resins, silica or silica-based materials including silicon and modified silicon, carbon, metals, inorganic glasses and plastics. The substrates may allow optical detection without appreciably fluorescing.

The substrate may be planar, although other configurations of substrates may be used as well. For example, probes may be placed on the inside surface of a tube, for flow-through sample analysis to minimize sample volume. Similarly, the substrate may be flexible, such as flexible foam, including closed cell foams made of particular plastics.

The substrate of the biochip and the probe may be derivatized with chemical functional groups for subsequent attachment of the two. For example, the biochip may be derivatized with a chemical functional group including, but not limited to, amino groups, carboxyl groups, oxo groups or thiol groups. Using these functional groups, the probes may be attached using functional groups on the probes either directly or indirectly using a linker.

The probes may be attached to the solid support by either the 5′ terminus, 3′ terminus, or via an internal nucleotide.

The probe may also be attached to the solid support non-covalently. For example, biotinylated oligonucleotides can be made, which may bind to surfaces covalently coated with streptavidin, resulting in attachment. Alternatively, probes may be synthesized on the surface using techniques such as photopolymerization and photolithography.

Diagnostics

A method of diagnosis is also provided. The method comprises detecting a differential expression level of AD nucleic acids in a biological sample. The sample may be derived from a patient. Diagnosis of AD, in a patient may allow for prognosis and selection of therapeutic strategy.

Kits

A kit is also provided and may comprise a nucleic acid described herein together with any or all of the following: assay reagents, buffers, probes and/or primers, and sterile saline or another pharmaceutically acceptable emulsion and suspension base. In addition, the kits may include instructional materials containing directions (e.g., protocols) for the practice of the methods described herein.

For example, the kit may be used for the amplification, detection, identification or quantification of a target nucleic acid sequence. The kit may comprise a poly(T) primer, a forward primer, a reverse primer, and a probe.

Any of the compositions described herein may be comprised in a kit. In a non-limiting example, reagents for isolating miRNA, labeling miRNA, and/or evaluating a miRNA population using an array are included in a kit. The kit may further include reagents for creating or synthesizing miRNA probes. The kits will thus comprise, in suitable container means, an enzyme for labeling the miRNA by incorporating labeled nucleotide or unlabeled nucleotides that are subsequently labeled. It may also include one or more buffers, such as reaction buffer, labeling buffer, washing buffer, or a hybridization buffer, compounds for preparing the miRNA probes, components for in situ hybridization and components for isolating miRNA. Other kits of the invention may include components for making a nucleic acid array comprising miRNA, and thus, may include, for example, a solid support.

The following examples are presented in order to more fully illustrate some embodiments of the invention. They should, in no way be construed, however, as limiting the broad scope of the invention.

### EXAMPLES

### Example 1

Methods

Patient and control's cohort

Serum samples from 27 patients with AD (medium/late stage) (Proteogenix Moscow) and 27 matched controls were collected.

Serum isolation and storage

8 ml of blood was collected from each individual directly into serum collection tubes (Greiner Bio-one, VACUETTE® Serum Tubes 455071). The whole blood was allowed to stand for about lh at room temperature (RT) before being centrifuged at 1800 g for 10 minutes at RT. The resultant serum was aliquoted into eppendorf tubes and stored at −80° C.

RNA Extraction

Serum (1000 was incubated over night at 57° C. with 300 μl pre-heated Proteinase K extraction solution as detailed in Table 3:

TABLE 1
Proteinase K extraction solution

| | | | Final in 400 ul |
|---|---|---|---|
| Component | Stock | Final | (serum + buffer) |
| NaCl | 5M | 0.118M | 0.0885M |
| Tris Hcl pH8 | 1M | 12 mM | 9 mM |
| DTT | 1M | 47 mM | 32.25 mM |
| EDTA pH8 | 0.5M | 12 mM | 9 mM |
| SDS | 20% (690 mM) | 2.4% (82.8 mM) | 1.8% (62.1 mM) |
| Proteinase K ABI | 20 mg/ml | 2.66 mg/ml | 2 mg/ml |
| (20 mg/ml) | | | |
| DDW | | | |

Followed by acid phenol:chloroform extraction, linear acrylamide (8 μl) was added. RNA was ETOH precipitated ON at −20° C. and re-suspend with DDW (43 μl). Next, DNase (Ambion) treatment was performed to eliminate residual DNA fragments. Finally, after a second acid phenol:chloroform extraction, the pellet was re-suspended in DDW.

qRT-PCR

RNA was subjected to a polyadenylation reaction as described previously (Shi, R. and Chiang, V. L. 2005, Biotechniques. 39(4):519-25). Briefly, RNA was incubated in the presence of poly (A) polymerase (PAP; NEB-M0276L), MnCl2, and ATP for 1 h at 37° C. Then, using an oligodT primer harboring a consensus sequence (complementary to the reverse primer) reverse transcription was performed on total RNA using SuperScript II RT (Invitrogen). Next, the cDNA was amplified by real time PCR; this reaction contained a microRNA-specific forward primer, a TaqMan probe complementary to the 3′ of the specific microRNA sequence as well as to part of the polyA adaptor sequence, and a universal reverse primer complementary to the consensus 3′ sequence of the oligodT tail.

The negative controls studied along the RNA samples, serve to detect potential contaminations and/or non-specific amplifications. The cycle number at which the fluorescence passes the threshold (Cycle Threshold—Ct) was measured for each miRNA in each sample.

Data Analysis and Statistics

The expression levels of 282 miRs were measured in each sample and normalized by scaling using 42 miRs.

Each sample was normalized by subtracting the average Ct of all miRs of the sample from the Ct of each miR, and adding back a scaling constant (the average Ct over the entire sample set). Normalized signals were compared between groups of in order to find miRs which can be used to differentiate between the groups. Significance of differences was assessed by a two-sided unpaired t-test. The Benjamini-Hochberg False Discovery Rate (FDR) method (Benjamini et al., 1995, J. Roy. Statist. Soc. Ser. B 57 no.1, 289-300) was used to control for multiple hypotheses testing, using an FDR of 0.01. Fold-change was calculated as 2^{x} where x is the absolute difference in median values of the normalized Ct in the two groups.

For the box plots, inverted-normalized signals were used such that high values represent high expression. The inverted-normalized signal for each miR is calculated by subtracting the normalized CT from 50.

### Example 2

Specific microRNAs are used for the Detection of AD in Serum Samples

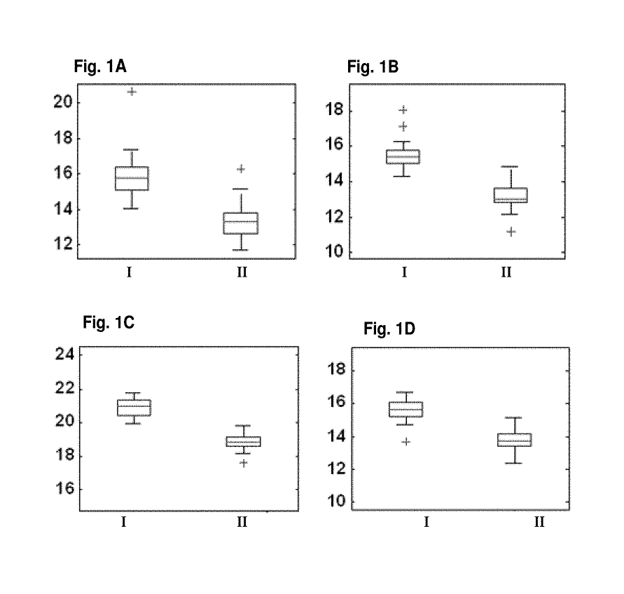

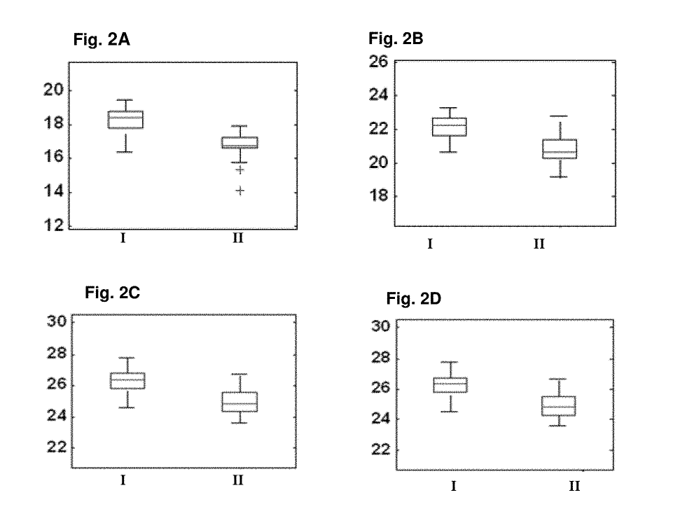

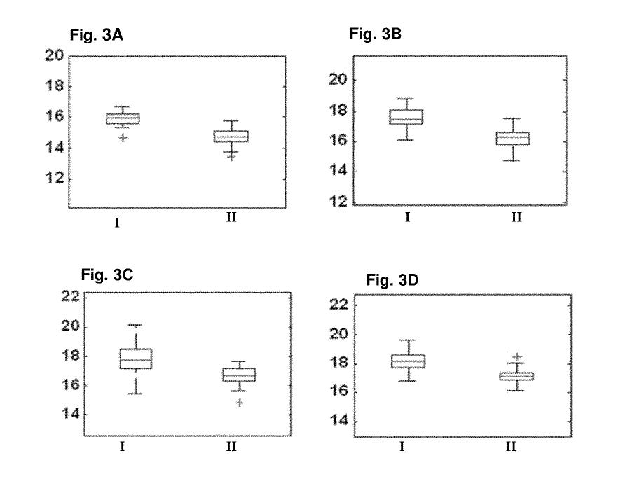

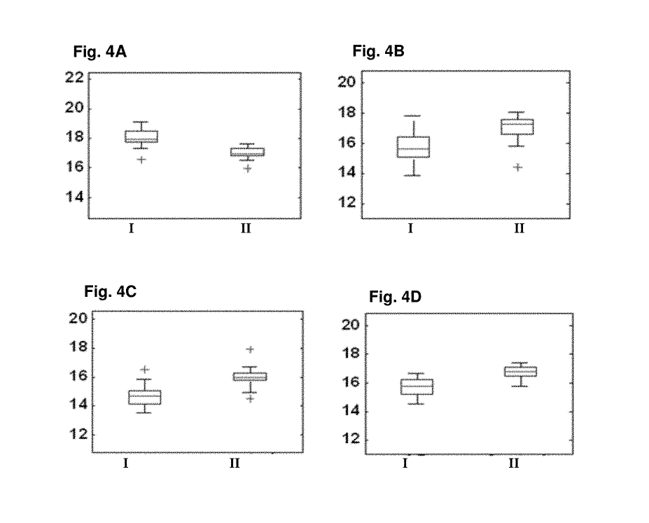

The levels of 282 microRNAs were measured by RT-PCR in all of the serum samples and normalized as described in Example 1. The signals for the 27 samples in the AD group were compared to the signals of the 27 samples in the Control group. The results are demonstrated in FIGS. 1-4 and Tables 2a and 2b.

TABLE 2a
Up-regulated miRs in AD group vs. Control group

| | | fold- | | | miR SEQ |
|---|---|---|---|---|---|
| miR name | p-value | change | median values | | ID NO: |
| hsa-miR-1296 | 7.90E−11 | 5.53 (+) | 15.794 | 13.326 | 1 |
| hsa-miR-424* | 1.30E−14 | 5.13 (+) | 15.389 | 13.031 | 2 |
| hsa-miR-424 | 5.70E−21 | 4.52 (+) | 20.996 | 18.819 | 3 |
| hsa-miR-629 | 1.00E−13 | 3.62 (+) | 15.598 | 13.74 | 4 |
| hsa-miR-143 | 8.30E−09 | 3.12 (+) | 18.394 | 16.75 | 5 |
| MID-16758 | 7.80E−08 | 2.96 (+) | 22.229 | 20.662 | 6 |
| MID-18395 | 2.70E−09 | 2.90 (+) | 26.371 | 24.834 | 7 |
| MID-16748 | 4.00E−09 | 2.80 (+) | 26.338 | 24.854 | 8 |
| hsa-miR-361-5p | 9.40E−13 | 2.37 (+) | 15.985 | 14.741 | 9 |
| hsa-miR-197 | 1.70E−10 | 2.20 (+) | 17.426 | 16.29 | 10 |
| MID-16582 | 3.40E−06 | 2.13 (+) | 17.793 | 16.702 | 11 |
| hsa-miR-148a | 3.30E−08 | 2.05 (+) | 18.173 | 17.138 | 12 |
| hsa-miR-145 | 2.40E−11 | 2.02 (+) | 17.971 | 16.959 | 13 |
| hsa-miR-200c | 1.80E−16 | 22.88 (+) | 16.284 | 11.768 | 14 |
| hsa-miR-659 | 2.10E−18 | 19.46 (+) | 17.385 | 13.102 | 15 |
| hsa-miR-181d | 1.40E−17 | 9.54 (+) | 18.897 | 15.642 | 16 |
| hsa-miR-370 | 1.60E−12 | 9.11 (+) | 19.125 | 15.938 | 17 |
| hsa-miR-139-3p | 2.60E−13 | 9.06 (+) | 15.834 | 12.655 | 18 |
| hsa-miR-483-5p | 1.80E−11 | 8.59 (+) | 16.51 | 13.408 | 19 |
| hsa-miR-127-5p | 4.60E−13 | 6.86 (+) | 15.461 | 12.683 | 20 |
| hsa-miR-1301 | 5.20E−12 | 6.13 (+) | 18.66 | 16.044 | 21 |
| hsa-miR-500a | 9.90E−13 | 5.61 (+) | 16.284 | 13.797 | 22 |
| hsa-miR-328 | 5.20E−10 | 4.90 (+) | 19.769 | 17.478 | 23 |
| hsa-miR-658 | 3.60E−08 | 4.55 (+) | 16.4 | 14.214 | 24 |
| hsa-miR-125a-3p | 6.20E−08 | 4.25 (+) | 18.021 | 15.935 | 25 |
| hsa-miR-433 | 1.00E−10 | 3.74 (+) | 16.509 | 14.607 | 26 |
| hsa-miR-124 | 7.90E−06 | 3.40 (+) | 17.447 | 15.681 | 27 |
| hsa-miR-671-5p | 1.40E−08 | 3.19 (+) | 21.043 | 19.371 | 28 |
| MID-15867 | 8.70E−22 | 25.49 (+) | 25.257 | 20.586 | 29 |
| MID-00690 | 1.40E−17 | 20.71 (+) | 15.046 | 10.674 | 30 |
| MID-17576 | 5.50E−10 | 17.95 (+) | 22.308 | 18.143 | 31 |
| MID-00064 | 1.70E−16 | 16.03 (+) | 23.478 | 19.475 | 32 |
| hsa-miR-298 | 2.50E−16 | 14.05 (+) | 15.216 | 11.403 | 33 |
| hsa-miR-675 | 2.90E−14 | 10.89 (+) | 18.116 | 14.671 | 34 |
| hsa-miR-765 | 1.70E−16 | 9.84 (+) | 16.09 | 12.791 | 35 |
| hsa-miR-3651 | 2.30E−13 | 5.96 (+) | 17.861 | 15.286 | 36 |
| hsa-miR-935 | 6.10E−11 | 4.32 (+) | 15.016 | 12.904 | 37 |
| hsa-miR-769-5p | 2.10E−08 | 4.08 (+) | 14.69 | 12.66 | 38 |
| hsa-miR-346 | 5.20E−10 | 3.99 (+) | 14.767 | 12.771 | 39 |
| hsa-miR-542-5p | 7.70E−08 | 3.34 (+) | 13.919 | 12.181 | 40 |
| hsa-miR-421 | 6.80E−08 | 2.95 (+) | 17.851 | 16.293 | 41 |
| hsa-miR-665 | 4.80E−11 | 2.94 (+) | 18.898 | 17.342 | 42 |
| hsa-miR-766 | 4.20E−08 | 2.92 (+) | 17.536 | 15.989 | 43 |
| hsa-miR-1180 | 9.90E−08 | 2.78 (+) | 21.692 | 20.215 | 44 |
| hsa-miR-339-3p | 1.10E−08 | 2.71 (+) | 15.968 | 14.53 | 45 |
| hsa-miR-500a* | 4.50E−12 | 2.69 (+) | 16.684 | 15.256 | 46 |
| hsa-miR-214 | 2.40E−08 | 2.64 (+) | 14.159 | 12.761 | 47 |
| hsa-miR-193a-5p | 7.40E−08 | 2.51 (+) | 17.329 | 16.001 | 48 |
| hsa-miR-345 | 9.00E−09 | 2.49 (+) | 16.706 | 15.391 | 49 |
| hsa-miR-106b* | 4.60E−08 | 2.34 (+) | 15.387 | 14.16 | 50 |
| hsa-miR-93* | 2.50E−08 | 2.22 (+) | 15.635 | 14.482 | 51 |

TABLE 2b
Down-regulated miRs in AD group vs. Control group

| | | fold- | | | miR SEQ |
|---|---|---|---|---|---|
| miR name | p-value | change | median values | | ID NO: |
| hsa-miR-199a-5p | 1.30E−06 | 3.23 (−) | 15.639 | 17.332 | 52 |
| hsa-miR-151-3p | 5.80E−10 | 2.53 (−) | 14.629 | 15.965 | 53 |
| hsa-miR-151-5p | 7.90E−09 | 2.06 (−) | 15.725 | 16.77 | 54 |
| hsa-miR-185 | 2.50E−10 | 1.81 (−) | 18.279 | 19.138 | 55 |
| hsa-miR-28-5p | 7.70E−06 | 1.61 (−) | 16.833 | 17.517 | 56 |
| hsa-miR-342-3p | 1.90E−06 | 1.58 (−) | 15.496 | 16.156 | 57 |
| hsa-miR-18b | 2.10E−06 | 1.54 (−) | 16.757 | 17.381 | 58 |
| hsa-miR-376c | 1.00E−06 | 6.13 (−) | 9.0621 | 11.677 | 59 |
| hsa-miR-409-3p | 1.30E−05 | 5.76 (−) | 10.973 | 13.498 | 60 |
| hsa-miR-382 | 5.90E−05 | 4.95 (−) | 11.114 | 13.421 | 61 |
| hsa-miR-495 | 6.50E−07 | 4.71 (−) | 12.039 | 14.274 | 62 |
| hsa-miR-375 | 9.10E−07 | 3.92 (−) | 12.98 | 14.95 | 63 |
| hsa-miR-154 | 1.50E−04 | 3.28 (−) | 12.645 | 14.359 | 64 |
| hsa-miR-146b-5p | 3.50E−05 | 2.15 (−) | 16.306 | 17.408 | 65 |
| hsa-miR-146a | 3.30E−05 | 1.89 (−) | 16.22 | 17.135 | 66 |
| hsa-let-7d | 5.20E−05 | 1.58 (−) | 16.924 | 17.584 | 67 |

TABLE 3a
Sequences of primers used for the detection of differential miRs

| | | FWD |
|---|---|---|
| | | SEQ ID |
| mir_name | FWD Sequence | NO: |
| hsa-miR-1296 | CAGTCATTTGGCTTAGGGCCCTGGCTCC | 68 |
| hsa-miR-424* | CAAAACGTGAGGCGCTGCTAT | 69 |
| hsa-miR-424 | CAGTCATTTGGCCAGCAGCAATTCATGT | 70 |
| hsa-miR-629 | CAGTCATTTGGCTGGGTTTACGTTGGGA | 71 |
| hsa-miR-143 | CAGTCATTTGGCTGAGATGAAGCACTGT | 72 |
| MID-16758 | GCATCCTGTTCGTGACGCCA | 73 |
| MID-18395 | GCGAATCCCACTTCTGACACCA | 74 |
| MID-16748 | GGCATCCCACTCCTGACACCA | 75 |
| hsa-miR-361-5p | CAGTCATTTGGCTTATCAGAATCTCCAG | 76 |
| hsa-miR-197 | CAGTCATTTGGCTTCACCACCTTCTCCA | 77 |
| MID-16582 | TTGGCAGTGAAGCATTGGACTGTA | 78 |
| hsa-miR-148a | CAGTCATTTGGCTCAGTGCACTACAGAA | 79 |
| hsa-miR-145 | CAGTCATTTGGCGTCCAGTTTTCCCAGG | 80 |
| hsa-miR-200c | CAGTCATTTGGGTAATACTGCCGGGTAA | 81 |
| hsa-miR-659 | CAGTCATTTGGCCTTGGTTCAGGGAGGG | 82 |
| hsa-miR-181d | GCAACATTCATTGTTGTCGGTGGGT | 83 |
| hsa-miR-370 | CAGTCATTTGGCGCCTGCTGGGGTGGAA | 84 |
| hsa-miR-139-3p | GGAGACGCGGCCCTGTTGGAGT | 85 |
| hsa-miR-483-5p | CAGTCATTTGGCAAGACGGGAGGAAAGA | 86 |
| hsa-miR-127-5p | CCTGAAGCTCAGAGGGCTCTGAT | 87 |
| hsa-miR-1301 | TTGCAGCTGCCTGGGAGTG | 88 |
| hsa-miR-500a | GCTAATCCTTGCTACCTGGGTGAGA | 89 |
| hsa-miR-328 | CAGTCATTTGGCCTGGCCCTCTCTGCCC | 90 |
| hsa-miR-658 | CAGTCATTTGGCGGCGGAGGGAAGTAGG | 91 |
| hsa-miR-125a- | ACAGGTGAGGTTCTTGGGAGCC | 92 |
| 3p | | |
| hsa-miR-433 | ATCATGATGGGCTCCTCGGTGT | 93 |
| hsa-miR-124 | CAGTCATTTGGCTAAGGCACGCGGTGAA | 94 |
| hsa-miR-671-5p | AGGAAGCCCTGGAGGGGCTGGAG | 95 |
| MID-15867 | CACATGAAAAGGGGAGAGGGCA | 96 |
| MID-00690 | GCTGGAGAAGACTGGAGAGGGTAT | 97 |
| MID-17576 | CCCAGGCTGGAGTGTAGTGGCGTGATCT | 98 |
| MID-00064 | AACTGGGGCGGGAAGGGGGAAG | 99 |
| hsa-miR-298 | CAGTCATTTGGCAGCAGAAGCAGGGAGG | 100 |
| hsa-miR-675 | TGGTGCGGAGAGGGCCCACAGTG | 101 |
| hsa-miR-765 | CAGTCATTTGGCTGGAGGAGAAGGAAGG | 102 |
| hsa-miR-3651 | CATAGCCCGGTCGCTGGTA | 103 |
| hsa-miR-935 | CCAGTTACCGCTTCCGCTACCGC | 104 |
| hsa-miR-769-5p | TGAGACCTCTGGGTTCTGAGCT | 105 |
| hsa-miR-346 | CAGTCATTTGGCTGTCTGCCCGCATGCC | 106 |
| hsa-miR-542-5p | TCGGGGATCATCATGTCACGAG | 107 |
| hsa-miR-421 | GCATCAACAGACATTAATTGGGCGC | 108 |
| hsa-miR-665 | ACCAGGAGGCTGAGGCCCCT | 109 |
| hsa-miR-766 | CAGTCATTTGGCACTCCAGCCCCACAGC | 110 |
| hsa-miR-1180 | TTTCCGGCTCGCGTGGGT | 111 |
| hsa-miR-339-3p | TGAGCGCCTCGACGACAGAGCCG | 112 |
| hsa-miR-500a* | CAGTCATTTGGCATGCACCTGGGCAAGG | 113 |
| hsa-miR-214 | CAGTCATTTGGGACAGCAGGCACAGACA | 114 |
| hsa-miR-193a- | CAGTCATTTGGCTGGGTCTTTGCGGGCG | 115 |
| 5p | | |
| hsa-miR-345 | CAGTCATTTGGCGCTGACTCCTAGTCCA | 116 |
| hsa-miR-106b* | CCGCACTGTGGGTACTTGCTGC | 117 |
| hsa-miR-93* | ACTGCTGAGCTAGCACTTCCCG | 118 |
| hsa-miR-199a- | CAGTCATTTGGGCCCAGTGTTCAGACTA | 119 |
| 5p | | |
| hsa-miR-151-3p | CAGTCATTTGGCCTAGACTGAAGCTCCT | 120 |
| hsa-miR-151-5p | CAGTCATTTGGGTCGAGGAGCTCACAGT | 121 |
| hsa-miR-185 | CAGTCATTTGGCTGGAGAGAAAGGCAGT | 122 |
| hsa-miR-28-5p | CAGTCATTTGGCAAGGAGCTCACAGTCT | 123 |
| hsa-miR-342-3p | CAGTCATTTGGGTCTCACACAGAAATCG | 124 |
| hsa-miR-18b | CAGTCATTTGGCTAAGGTGCATCTAGTG | 125 |
| hsa-miR-376c | CAGTCATTTGGCAACATAGAGGAAATTC | 126 |
| hsa-miR-409-3p | CAGTCATTTGGCGAATGTTGCTCGGTGA | 127 |
| hsa-miR-382 | CAGTCATTTGGCGAAGTTGTTCGTGGTG | 128 |
| hsa-miR-495 | CAGTCATTTGGCAAACAAACATGGTGCA | 129 |
| hsa-miR-375 | CAGTCATTTGGGTTTGTTCGTTCGGCTC | 130 |
| hsa-miR-154 | CAGTCATTTGGCTAGGTTATCCGTGTTG | 131 |
| hsa-miR-146b- | CAGTCATTTGGCTGAGAACTGAATTCCA | 132 |
| 5p | | |
| hsa-miR-146a | CAGTCATTTGGCTGAGAACTGAATTCCA | 133 |
| hsa-let-7d | CAGTCATTTGGCAGAGGTAGTAGGTTGC | 134 |

TABLE 3b
Sequences of probes used for the detection of differential miRs

| | | MGB SEQ ID |
|---|---|---|
| miR name | MGB Sequence | NO: |
| hsa-miR-1296 | CCGTTTTTTTTTTTTGGAGATGG | 135 |
| hsa-miR-424* | AAAACCGATAGTGAGTCG | 136 |
| hsa-miR-424 | CCGTTTTTTTTTTTTCAAAACAT | 137 |
| hsa-miR-629 | CCGTTTTTTTTTTTTAGTTCTCC | 138 |
| hsa-miR-143 | CCGTTTTTTTTTTTTGAGCTACA | 139 |
| MID-16758 | AAAACCGATAGTGAGTCG | 140 |
| MID-18395 | AAAACCGATAGTGAGTCG | 141 |
| MID-16748 | AAAACCGATAGTGAGTCG | 142 |
| hsa-miR-361-5p | CCGTTTTTTTTTTTTGTACCCCT | 143 |
| hsa-miR-197 | CCGTTTTTTTTTTTTGCTGGGTG | 144 |
| MID-16582 | AAAACCGATAGTGAGTCG | 145 |
| hsa-miR-148a | CCGTTTTTTTTTTTTACAAAGTT | 146 |
| hsa-miR-145 | CCGTTTTTTTTTTTTAGGGATTC | 147 |
| hsa-miR-200c | CGTTTTTTTTTTTTCCATCATT | 148 |
| hsa-miR-659 | CGTTTTTTTTTTTTGGGGACCC | 149 |
| hsa-miR-181d | AAAACCGATAGTGAGTCG | 150 |
| hsa-miR-370 | TCCGTTTTTTTTTTTTACCAGGTT | 151 |
| hsa-miR-139-3p | AAAACCGATAGTGAGTCG | 152 |
| hsa-miR-483-5p | CCGTTTTTTTTTTTTCTCCCTTC | 153 |
| hsa-miR-127-5p | AAAACCGATAGTGAGTCG | 154 |
| hsa-miR-1301 | AAAACCGATAGTGAGTCG | 155 |
| hsa-miR-500a | AAAACCGATAGTGAGTCG | 156 |
| hsa-miR-328 | CCGTTTTTTTTTTTTACGGAAGG | 157 |
| hsa-miR-658 | CCGTTTTTTTTTTTTACCAACGG | 158 |
| hsa-miR-125a-3p | AAAACCGATAGTGAGTCG | 159 |
| hsa-miR-433 | AAAACCGATAGTGAGTCG | 160 |
| hsa-miR-124 | CCGTTTTTTTTTTTTGGCATTCA | 161 |
| hsa-miR-671-5p | AAAACCGATAGTGAGTCG | 162 |
| MID-15867 | AAAACCGATAGTGAGTCG | 163 |
| MID-00690 | AAAACCGATAGTGAGTCG | 164 |
| MID-17576 | AAAACCGATAGTGAGTCG | 165 |
| MID-00064 | AAAACCGATAGTGAGTCG | 166 |
| hsa-miR-298 | CCGTTTTTTTTTTTTGGGAGAAC | 167 |
| hsa-miR-675 | AAAACCGATAGTGAGTCG | 168 |
| hsa-miR-765 | CCGTTTTTTTTTTTTCATCACCT | 169 |
| hsa-miR-3651 | AAAACCGATAGTGAGTCG | 170 |
| hsa-miR-935 | AAAACCGATAGTGAGTCG | 171 |
| hsa-miR-769-5p | AAAACCGATAGTGAGTCG | 172 |
| hsa-miR-346 | CCGTTTTTTTTTTTTAGAGGCAG | 173 |
| hsa-miR-542-5p | AAAACCGATAGTGAGTCG | 174 |
| hsa-miR-421 | AAAACCGATAGTGAGTCG | 175 |
| hsa-miR-665 | AAAACCGATAGTGAGTCG | 176 |
| hsa-miR-766 | CCGTTTTTTTTTTTTGCTGAGGC | 177 |
| hsa-miR-1180 | AAAACCGATAGTGAGTCG | 178 |
| hsa-miR-339-3p | AAAACCGATAGTGAGTCG | 179 |
| hsa-miR-500a* | CGTTTTTTTTTTTTCAGAATCC | 180 |
| hsa-miR-214 | CCGTTTTTTTTTTTTACTGCCTG | 181 |
| hsa-miR-193a-5p | CCGTTTTTTTTTTTTCATCTCGC | 182 |
| hsa-miR-345 | CGTTTTTTTTTTTTGAGCCCTG | 183 |
| hsa-miR-106b* | AAAACCGATAGTGAGTCG | 184 |
| hsa-miR-93* | AAAACCGATAGTGAGTCG | 185 |
| hsa-miR-199a-5p | CCGTTTTTTTTTTTTGAACAGGT | 186 |
| hsa-miR-151-3p | CCGTTTTTTTTTTTTCCTCAAGG | 187 |
| hsa-miR-151-5p | CCGTTTTTTTTTTTTACTAGACT | 188 |
| hsa-miR-185 | CGTTTTTTTTTTTTCAGGAACT | 189 |
| hsa-miR-28-5p | CCGTTTTTTTTTTTTCTCAATAG | 190 |
| hsa-miR-342-3p | CCGTTTTTTTTTTTTACGGGTGC | 191 |
| hsa-miR-18b | CCGTTTTTTTTTTTTCTAACTGC | 192 |
| hsa-miR-376c | CCGTTTTTTTTTTTTACGTGGAA | 193 |
| hsa-miR-409-3p | CCGTTTTTTTTTTTTAGGGGTTC | 194 |
| hsa-miR-382 | CGTTTTTTTTTTTTCGAATCCA | 195 |
| hsa-miR-495 | CCGTTTTTTTTTTTTAAGAAGTG | 196 |
| hsa-miR-375 | CCGTTTTTTTTTTTTCACGCGAG | 197 |
| hsa-miR-154 | CCGTTTTTTTTTTTTCGAAGGCA | 198 |
| hsa-miR-146b-5p | CCGTTTTTTTTTTTTAGCCTATG | 199 |
| hsa-miR-146a | CCGTTTTTTTTTTTTAACCCATG | 200 |
| hsa-let-7d | CCGTTTTTTTTTTTTAACTATGC | 201 |
| Reverse primer | GCGAGCACAGAATTAATACGAC | 202 |

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It should be understood that the detailed description and specific examples, while indicating preferred embodiments of the invention, are given by way of illustration only, since various changes and modifications within the spirit and scope of the invention will become apparent to those skilled in the art from this detailed description.

1. A method of diagnosing Alzheimer's disease (AD) in a subject, the method comprising: obtaining a biological sample from a subject; determining an expression profile in said sample of a nucleic acid sequence selected from the group consisting of SEQ ID NOS: 1-67; a fragment thereof or a sequence having at least about 80% identity thereto; and comparing said expression profile to a reference expression profile wherein a difference in the level of expression profile in at least one or more nucleic acid sequence in said biological sample compared to said reference expression profile is diagnostic for AD.

2. The method of claim 1, wherein relatively high expression levels of a nucleic acid sequence selected from the group consisting of SEQ ID NOS: 1-51; a fragment thereof and a sequence having at least about 80% identity thereto is diagnostic for AD.

3. The method of claim 1, wherein relatively low expression levels of a nucleic acid sequence selected from the group consisting of SEQ ID NOS: 52-67; a fragment thereof and a sequence having at least about 80% identity thereto is diagnostic for AD.

4. The method of claim 1, wherein said biological sample is selected from the group consisting of bodily fluid, a cell line and a tissue sample.

5. The method of claim 4, wherein said bodily fluid sample is a serum sample.

6. The method of claim 4, wherein said bodily fluid sample is a blood sample.

7. The method of claim 1, wherein the method comprises determining the expression levels of at least two nucleic acid sequences.

8. The method of claim 7, wherein the method further comprises combining one or more expression ratios of said nucleic acid sequences.

9. The method of claim 1, wherein the expression levels are determined by a method selected from the group consisting of nucleic acid hybridization, nucleic acid amplification, and a combination thereof.

10. The method of claim 9, wherein the nucleic acid amplification method is real-time PCR.

11. The method of claim 10, wherein the real-time PCR method comprises forward and reverse primers.

12. The method of claim 11, wherein the forward primer comprises a sequence selected from the group consisting of SEQ ID NOS: 68-134; a fragment thereof and a sequence having at least about 80% identity thereto.

13. The method of claim 12, wherein the real-time PCR method further comprises a probe.

14. The method of claim 13, wherein the probe comprises a nucleic acid sequence that is complementary to a sequence selected from the group consisting of SEQ ID NOS: 1-67; a fragment thereof and a sequence having at least about 80% identity thereto.

15. The method of claim 14, wherein the probe comprises a nucleic acid sequence selected from the group consisting of SEQ ID NOS: 135-201; a fragment thereof and a sequence having at least about 80% identity thereto.

16. The method of claim 11, wherein the reverse primer comprises SEQ ID NO: 202, a fragment thereof and a sequence having at least about 80% identity thereto.

17. A kit for assessing AD in a subject, said kit comprising a probe comprising a nucleic acid sequence that is complementary to a sequence selected from the group consisting of SEQ ID NOS: 1-67; a fragment thereof and a sequence having at least about 80% identity thereto.

18. The kit of claim 17, wherein the probe comprises a nucleic acid sequence selected from the group consisting of SEQ ID NOS: 135-201; a fragment thereof and a sequence having at least about 80% identity thereto.

19. The kit of claim 17, wherein the kit further comprises a forward primer comprising a sequence selected from the group consisting of any one of SEQ ID NOS: 68-134; a fragment thereof and a sequence having at least about 80% identity thereto.

20. The kit of claim 17, wherein the kit further comprises a reverse primer comprises SEQ ID NO: 202, a fragment thereof and a sequence having at least about 80% identity thereto.

21. The method of claim 1, further comprising managing subject treatment based on the AD status.

22. The method of claim 21, wherein managing subject treatment is selected from ordering further diagnostic tests and administering at least one therapeutic agent.
